SEQUENCE LISTING (339 sequences)

SEQ ID NO: 1 - protein, 120 aa, Artificial Sequence
  source: /note=“Description of Artificial Sequence Synthetic polypeptide”
QVQLQESGGGLVQAGGSLRLSCAASGSIFSRDTMAWYRQVPGEQRELVAFITNFGGTNHADSVKGRFTISRDNAKNTVYLQMNSLKPEDTAVYYCAAGLVSLRTHTWEYWGQGTQVTVSS

SEQ ID NO: 2 - protein, 122 aa, Artificial Sequence
  source: /note=“Description of Artificial Sequence Synthetic polypeptide”
QVQLQESGGGLVQAGDSLSLSCVDSKRTADRYVMHWFRQVPGKDREFVAAISGNGLVRNYADSVKGRFTISRDNAKNMVYLQMKSLKPEDTAVYYCAADFVLDSRWRYVPYWGQGTQVTVSS

SEQ ID NO: 3 - protein, 120 aa, Artificial Sequence
  source: /note=“Description of Artificial Sequence Synthetic polypeptide”
QVQLQESGGGLVQAGGSLRLSCAASGSIFSRDTMAWYRQVPGEQRELVAYITNFGGTNHADSVKGRFTISRDNAKNTVYLQMNSLKPEDTAVYYCAAGLVSLHTHTWEYWGQGTQVTVSS

SEQ ID NO: 4 - protein, 122 aa, Artificial Sequence
  source: /note=“Description of Artificial Sequence Synthetic polypeptide”
QVQLQESGGGLVQAGGSLRLSCAASGSIFSINAMGWYRQAPGKQRELVAGITSGGSTNYADSVKGRFTISRDNAKNTVYLQMNSLKPEDTAVYYCAADYVGLGDDAFYVPYWGQGTQVTVSA

SEQ ID NO: 5 - protein, 115 aa, Artificial Sequence
  source: /note=“Description of Artificial Sequence Synthetic polypeptide”
QVQLQQSGGGLVQPGGSLRLSCAASRSIRSADAMAWYRQAPGKQRVWVATITSGGSTYYTDSVKGRFTISRDNAKSTVYLQMSSLKPEDTAVYYCNAVGDRTSYWGQGTQVTVAS

SEQ ID NO: 6 - protein, 128 aa, Artificial Sequence
  source: /note=“Description of Artificial Sequence Synthetic polypeptide”
QVQLQESGGGLVQTGGSLRLSCAASGRTFTRNAMGWFRQAPGKAREFVAVISWSGNRAYSDSVKGRFTISRDNGKNLVSLQMNSLKPEDTAVYYCAAAREAITSTYYTPHVLTDYDAWGQGAQVTVSS

SEQ ID NO: 7 - protein, 128 aa, Artificial Sequence
  source: /note=“Description of Artificial Sequence Synthetic polypeptide”
QVQLQESGGGLVQPGGSLRLSCAASGRTFRRNAMGWFRQAPGKAREFVATISWSGNRAYSDSVKGRFTISRDNGKNLVSLQMNSLKPEDTAVYYCAAARERITSTYYTPHVLTDYDAWGQGAQVTVSS

SEQ ID NO: 8 - protein, 120 aa, Artificial Sequence
  source: /note=“Description of Artificial Sequence Synthetic polypeptide”
QVQLQESGGGLVQAGGSLRLSCAASGSIFSRDTMAWYRQVPGEQRELVAYITNFGGTNHAGSVKGRFTISRDNAKNTVYLQMNSLKPEDTAVYYCAAELVSLRTHTWEYWGQGTQVTVSS

SEQ ID NO: 9 - protein, 128 aa, Artificial Sequence
  source: /note=“Description of Artificial Sequence Synthetic polypeptide”
QVQLQQSGGGLVQAGGSLRLSCAASGRTFSRNAMGWFRQAPGKAREFVATISWSGNRAYSDSVKGRFTISRDNEKNLVSLHMNSLKPEDTAVYYCAAARERITSTYYTPHVLTDYDAWGQGAQVTVSS

SEQ ID NO: 10 - protein, 121 aa, Artificial Sequence
  source: /note=“Description of Artificial Sequence Synthetic polypeptide”
QVQLQESGGGLVQAGGSLSLSCTASGRTFSTFPVAWFRQAPGKEREFVARINWFTTDTYYADSVKGRFTISRDSAKNTVYLQMNALKPEDTAIYYCAAARSNTGTSRFDYWGQGTQVTVSS

SEQ ID NO: 11 - protein, 119 aa, Artificial Sequence
  source: /note=“Description of Artificial Sequence Synthetic polypeptide”
QVQLQESGGGLVQAGGSLRLSCAASTNIATFTTMGWYRQAPGKERELVATTTPWGATTSYADSVKGRFTISNDNAKNTVNLQMNSLKPEDTAVYYCNAQQDIPTTQTYWGQGTQVTVSS

SEQ ID NO: 12 - protein, 124 aa, Artificial Sequence
  source: /note=“Description of Artificial Sequence Synthetic polypeptide”
QVQLQESGGGLVQAGGSLRLSCAANGRTFSSYAMAWFRQAPGKEREFVAAVGWSGGRTYYTDSVKGRFTISRDNAKDTVYLQMNSLKPEDTAVYYCAATRGWAQATLLYDYDYWGQGTQVTVSS

SEQ ID NO: 13 - protein, 123 aa, Artificial Sequence
  source: /note=“Description of Artificial Sequence Synthetic polypeptide”
QVQLQQSGGGLVQAGGSVRLSCTANGLTFGNYAMAWFRRTPGKERAFVGGMSASGAGTYYLDSVKGRFTISRDTAKNTVYLEMNSLKAEDTAVYYCAANSIYPGRRWASYDYWGQGTQVTVSS

SEQ ID NO: 14 - protein, 123 aa, Artificial Sequence
  source: /note=“Description of Artificial Sequence Synthetic polypeptide”
QVQLQQSGGGLVQAGGSVRLSCAASGLTLGNYALAWFRRTPGKEREFVAGMSGSGAGTYYLDSVNGRFTISRDNAKNTLYLQMNSLKAEDTAVYYCAANSIYPGRRWASYDYWGQGTQVTVSS

SEQ ID NO: 15 - protein, 120 aa, Artificial Sequence
  source: /note=“Description of Artificial Sequence Synthetic polypeptide”
QVQLQESGGGLVQAGGSLRLSCAASASIFSRDTMAWYRQVPGEQRELVAYVTNFGGTNHADSVKGRFTISRDNAKNTVYLQMNSLKPEDTAVYYCAADLISLRTHTWEYWGQGTQVTVSS

SEQ ID NO: 16 - protein, 120 aa, Artificial Sequence
  source: /note=“Description of Artificial Sequence Synthetic polypeptide”
QVQLQESGGGLVQPGGSLRVSCAASGFTFSRDTMAWYRQVPGEQRELVAYITNFGGTNHADSVKGRFTISRDNAKNTVYLQMNSLKPEDTAVYYCAAGLVSLRTHTWEYWGQGTQVTVSS

SEQ ID NO: 17 - protein, 128 aa, Artificial Sequence
  source: /note=“Description of Artificial Sequence Synthetic polypeptide”
QVQLQESGGGLVQTGGSLRLSCAASGRTFRRNAMGWFRQAPGKAREFVATITWSGNRAYSDSVKGRFTISRDNEKNLVSLQMNSLKPEDTAVYYCAAARERITSTYYTPHVLTEYDAWGQGAQVTVSS

SEQ ID NO: 18 - protein, 115 aa, Artificial Sequence
  source: /note=“Description of Artificial Sequence Synthetic polypeptide”
QVQLQQSGGGLVQPGGSLRLSCAASGSIFSVDAMAWYRQAPGKQREWVATFTKGGSTYYAGSVKGRFTISRDNAKNTVYLQMSSLKPEDTAVYYCNAVGDRTGAWGQGTQVTVAS

SEQ ID NO: 19 - protein, 120 aa, Artificial Sequence
  source: /note=“Description of Artificial Sequence Synthetic polypeptide”
QVQLQESGGGLVQAGGSLRLSCAASGSIFSRDTMAWYRQVPGEQRELVAYITNFGGTNHADSVKGRFTISRDNAKNTVYLQMNSLKPEETAVYYCAAGLVSLRTHTWEYWGQGTQVTVSS

SEQ ID NO: 20 - protein, 128 aa, Artificial Sequence
  source: /note=“Description of Artificial Sequence Synthetic polypeptide”
QVQLQESGGGLVQAGGSLRLSCAASGRTFVNYNMGWFRQAPGKEREFVATINWSGGSTYYAGSVKGRFTISRDSAKNTVYLQMNKLKPEDTAVYYCAAEVGYGYQGPPLTTPSMYDYWGQGTQVTVST

SEQ ID NO: 21 - protein, 120 aa, Artificial Sequence
  source: /note=“Description of Artificial Sequence Synthetic polypeptide”
QVQLQESGGGLVQAGGSLRLSCVASGSIFSRDTMAWYRQVPGEQRELVAYITNFGGTNHADSVKGRFTISRDNAKNTVYLQMNSLKPEDTAVYYCAAGLLSLRSHTWELWGQGTQVTVSS

SEQ ID NO: 22 - protein, 120 aa, Artificial Sequence
  source: /note=“Description of Artificial Sequence Synthetic polypeptide”
QVQLQESGGGLVQAGGSLRLSCATSGSIFSRDTMAWYRQVPGEQRELVAYITNFGGTNHADSVKGRFTISRDNAKNTVYLQMNSLKPEDTAVYYCAAGLLSLRTHTWEYWGQGTQVTVSS

SEQ ID NO: 23 - protein, 126 aa, Artificial Sequence
  source: /note=“Description of Artificial Sequence Synthetic polypeptide”
QVQLQESGGGLVQPGDSLRLSCAPSGRTFSGYVTGWFRQAPGMEREFVAAITWSGDSTYYADSVKGRFQISRDSAKNTVYLQMNSLKPEDAGVYYCAVKSQTYSTDYVQPRRYAYWGQGTQVTVSS

SEQ ID NO: 24 - protein, 120 aa, Artificial Sequence
  source: /note=“Description of Artificial Sequence Synthetic polypeptide”
QVQLQESGGGLVQPGGSLRLSCAVSGTIFSITPMGWYRQAPGKQRELVASISGGGSTNYTDPVKGRFTISRDKARNTVYLQMNNLKPEDTAVYYCNAASLAIVRGINNYWGQGTQVTVSS

SEQ ID NO: 25 - protein, 120 aa, Artificial Sequence
  source: /note=“Description of Artificial Sequence Synthetic polypeptide”
QVQLQESGGGLVQAGGSLRLSCAASGSIFSRDNMAWYRQVPGEQRELVALITNFGGTNHADSVKGRFTISRDNAKNTVYLQMNSLKPEDTAVYYCAAGLVSLLTHTWEYWGQGTQVTVSS

SEQ ID NO: 26 - protein, 9 aa, Artificial Sequence
  source: /note=“Description of Artificial Sequence Synthetic peptide”
GSIFSRDTM

SEQ ID NO: 27 - protein, 9 aa, Artificial Sequence
  source: /note=“Description of Artificial Sequence Synthetic peptide”
KRTADRYVM

SEQ ID NO: 28 - protein, 9 aa, Artificial Sequence
  source: /note=“Description of Artificial Sequence Synthetic peptide”
GSIFSRDTM

SEQ ID NO: 29 - protein, 9 aa, Artificial Sequence
  source: /note=“Description of Artificial Sequence Synthetic peptide”
GSIFSINAM

SEQ ID NO: 30 - protein, 9 aa, Artificial Sequence
  source: /note=“Description of Artificial Sequence Synthetic peptide”
RSIRSADAM

SEQ ID NO: 31 - protein, 9 aa, Artificial Sequence
  source: /note=“Description of Artificial Sequence Synthetic peptide”
GRTFTRNAM

SEQ ID NO: 32 - protein, 9 aa, Artificial Sequence
  source: /note=“Description of Artificial Sequence Synthetic peptide”
GRTFRRNAM

SEQ ID NO: 33 - protein, 9 aa, Artificial Sequence
  source: /note=“Description of Artificial Sequence Synthetic peptide”
GSIFSRDTM

SEQ ID NO: 34 - protein, 9 aa, Artificial Sequence
  source: /note=“Description of Artificial Sequence Synthetic peptide”
GRTFSRNAM

SEQ ID NO: 35 - protein, 9 aa, Artificial Sequence
  source: /note=“Description of Artificial Sequence Synthetic peptide”
GRTFSTFPV

SEQ ID NO: 36 - protein, 9 aa, Artificial Sequence
  source: /note=“Description of Artificial Sequence Synthetic peptide”
TNIATFTTM

SEQ ID NO: 37 - protein, 9 aa, Artificial Sequence
  source: /note=“Description of Artificial Sequence Synthetic peptide”
GRTFSSYAM

SEQ ID NO: 38 - protein, 9 aa, Artificial Sequence
  source: /note=“Description of Artificial Sequence Synthetic peptide”
GLTFGNYAM

SEQ ID NO: 39 - protein, 9 aa, Artificial Sequence
  source: /note=“Description of Artificial Sequence Synthetic peptide”
GLTLGNYAL

SEQ ID NO: 40 - protein, 9 aa, Artificial Sequence
  source: /note=“Description of Artificial Sequence Synthetic peptide”
ASIFSRDTM

SEQ ID NO: 41 - protein, 9 aa, Artificial Sequence
  source: /note=“Description of Artificial Sequence Synthetic peptide”
GFTFSRDTM

SEQ ID NO: 42 - protein, 9 aa, Artificial Sequence
  source: /note=“Description of Artificial Sequence Synthetic peptide”
GRTFRRNAM

SEQ ID NO: 43 - protein, 9 aa, Artificial Sequence
  source: /note=“Description of Artificial Sequence Synthetic peptide”
GSIFSVDAM

SEQ ID NO: 44 - protein, 9 aa, Artificial Sequence
  source: /note=“Description of Artificial Sequence Synthetic peptide”
GSIFSRDTM

SEQ ID NO: 45 - protein, 9 aa, Artificial Sequence
  source: /note=“Description of Artificial Sequence Synthetic peptide”
GRTFVNYNM

SEQ ID NO: 46 - protein, 9 aa, Artificial Sequence
  source: /note=“Description of Artificial Sequence Synthetic peptide”
GSIFSRDTM

SEQ ID NO: 47 - protein, 9 aa, Artificial Sequence
  source: /note=“Description of Artificial Sequence Synthetic peptide”
GSIFSRDTM

SEQ ID NO: 48 - protein, 9 aa, Artificial Sequence
  source: /note=“Description of Artificial Sequence Synthetic peptide”
GRTFSGYVT

SEQ ID NO: 49 - protein, 9 aa, Artificial Sequence
  source: /note=“Description of Artificial Sequence Synthetic peptide”
GTIFSITPM

SEQ ID NO: 50 - protein, 9 aa, Artificial Sequence
  source: /note=“Description of Artificial Sequence Synthetic peptide”
GSIFSRDNM

SEQ ID NO: 51 - protein, 7 aa, Artificial Sequence
  source: /note=“Description of Artificial Sequence Synthetic peptide”
ITNFGGT

SEQ ID NO: 52 - protein, 8 aa, Artificial Sequence
  source: /note=“Description of Artificial Sequence Synthetic peptide”
ISGNGLVR

SEQ ID NO: 53 - protein, 7 aa, Artificial Sequence
  source: /note=“Description of Artificial Sequence Synthetic peptide”
ITNFGGT

SEQ ID NO: 54 - protein, 7 aa, Artificial Sequence
  source: /note=“Description of Artificial Sequence Synthetic peptide”
ITSGGST

SEQ ID NO: 55 - protein, 7 aa, Artificial Sequence
  source: /note=“Description of Artificial Sequence Synthetic peptide”
ITSGGST

SEQ ID NO: 56 - protein, 7 aa, Artificial Sequence
  source: /note=“Description of Artificial Sequence Synthetic peptide”
ISWSGNR

SEQ ID NO: 57 - protein, 7 aa, Artificial Sequence
  source: /note=“Description of Artificial Sequence Synthetic peptide”
ISWSGNR

SEQ ID NO: 58 - protein, 7 aa, Artificial Sequence
  source: /note=“Description of Artificial Sequence Synthetic peptide”
ITNFGGT

SEQ ID NO: 59 - protein, 7 aa, Artificial Sequence
  source: /note=“Description of Artificial Sequence Synthetic peptide”
ISWSGNR

SEQ ID NO: 60 - protein, 8 aa, Artificial Sequence
  source: /note=“Description of Artificial Sequence Synthetic peptide”
INWFTTDT

SEQ ID NO: 61 - protein, 8 aa, Artificial Sequence
  source: /note=“Description of Artificial Sequence Synthetic peptide”
TTPWGATT

SEQ ID NO: 62 - protein, 8 aa, Artificial Sequence
  source: /note=“Description of Artificial Sequence Synthetic peptide”
VGWSGGRT

SEQ ID NO: 63 - protein, 8 aa, Artificial Sequence
  source: /note=“Description of Artificial Sequence Synthetic peptide”
MSGSGAGT

SEQ ID NO: 64 - protein, 8 aa, Artificial Sequence
  source: /note=“Description of Artificial Sequence Synthetic peptide”
MSGSGAGT

SEQ ID NO: 65 - protein, 7 aa, Artificial Sequence
  source: /note=“Description of Artificial Sequence Synthetic peptide”
VTNFGGT

SEQ ID NO: 66 - protein, 7 aa, Artificial Sequence
  source: /note=“Description of Artificial Sequence Synthetic peptide”
ITNFGGT

SEQ ID NO: 67 - protein, 7 aa, Artificial Sequence
  source: /note=“Description of Artificial Sequence Synthetic peptide”
ITWSGNR

SEQ ID NO: 68 - protein, 7 aa, Artificial Sequence
  source: /note=“Description of Artificial Sequence Synthetic peptide”
FTKGGST

SEQ ID NO: 69 - protein, 7 aa, Artificial Sequence
  source: /note=“Description of Artificial Sequence Synthetic peptide”
ITNFGGT

SEQ ID NO: 70 - protein, 8 aa, Artificial Sequence
  source: /note=“Description of Artificial Sequence Synthetic peptide”
INWSGGST

SEQ ID NO: 71 - protein, 7 aa, Artificial Sequence
  source: /note=“Description of Artificial Sequence Synthetic peptide”
ITNFGGT

SEQ ID NO: 72 - protein, 7 aa, Artificial Sequence
  source: /note=“Description of Artificial Sequence Synthetic peptide”
ITNFGGT

SEQ ID NO: 73 - protein, 8 aa, Artificial Sequence
  source: /note=“Description of Artificial Sequence Synthetic peptide”
ITWSGDST

SEQ ID NO: 74 - protein, 7 aa, Artificial Sequence
  source: /note=“Description of Artificial Sequence Synthetic peptide”
ISGGGST

SEQ ID NO: 75 - protein, 7 aa, Artificial Sequence
  source: /note=“Description of Artificial Sequence Synthetic peptide”
ITNFGGT

SEQ ID NO: 76 - protein, 15 aa, Artificial Sequence
  source: /note=“Description of Artificial Sequence Synthetic peptide”
AADFVLDSRWRYVPY

SEQ ID NO: 77 - protein, 15 aa, Artificial Sequence
  source: /note=“Description of Artificial Sequence Synthetic peptide”
AADFVLDSRWRYVPY

SEQ ID NO: 78 - protein, 14 aa, Artificial Sequence
  source: /note=“Description of Artificial Sequence Synthetic peptide”
AAGLVSLHTHTWEY

SEQ ID NO: 79 - protein, 16 aa, Artificial Sequence
  source: /note=“Description of Artificial Sequence Synthetic peptide”
AADYVGLGDDAFYVPY

SEQ ID NO: 80 - protein, 9 aa, Artificial Sequence
  source: /note=“Description of Artificial Sequence Synthetic peptide”
NAVGDRTSY

SEQ ID NO: 81 - protein, 22 aa, Artificial Sequence
  source: /note=“Description of Artificial Sequence Synthetic peptide”
AAAREAITSTYYTPHVLTDYDA

SEQ ID NO: 82 - protein, 22 aa, Artificial Sequence
  source: /note=“Description of Artificial Sequence Synthetic peptide”
AAARERITSTYYTPHVLTDYDA

SEQ ID NO: 83 - protein, 14 aa, Artificial Sequence
  source: /note=“Description of Artificial Sequence Synthetic peptide”
AAELVSLRTHTWEY

SEQ ID NO: 84 - protein, 22 aa, Artificial Sequence
  source: /note=“Description of Artificial Sequence Synthetic peptide”
AAARERITSTYYTPHVLTDYDA

SEQ ID NO: 85 - protein, 14 aa, Artificial Sequence
  source: /note=“Description of Artificial Sequence Synthetic peptide”
AAARSNTGTSRFDY

SEQ ID NO: 86 - protein, 12 aa, Artificial Sequence
  source: /note=“Description of Artificial Sequence Synthetic peptide”
NAQQDIPTTQTY

SEQ ID NO: 87 - protein, 17 aa, Artificial Sequence
  source: /note=“Description of Artificial Sequence Synthetic peptide”
AATRGWAQATLLYDYDY

SEQ ID NO: 88 - protein, 16 aa, Artificial Sequence
  source: /note=“Description of Artificial Sequence Synthetic peptide”
AANSIYPGRRWASYDY

SEQ ID NO: 89 - protein, 16 aa, Artificial Sequence
  source: /note=“Description of Artificial Sequence Synthetic peptide”
AANSIYPGRRWASYDY

SEQ ID NO: 90 - protein, 14 aa, Artificial Sequence
  source: /note=“Description of Artificial Sequence Synthetic peptide”
AADLISLRTHTWEY

SEQ ID NO: 91 - protein, 14 aa, Artificial Sequence
  source: /note=“Description of Artificial Sequence Synthetic peptide”
AAGLVSLRTHTWEY

SEQ ID NO: 92 - protein, 22 aa, Artificial Sequence
  source: /note=“Description of Artificial Sequence Synthetic peptide”
AAARERITSTYYTPHVLTEYDA

SEQ ID NO: 93 - protein, 9 aa, Artificial Sequence
  source: /note=“Description of Artificial Sequence Synthetic peptide”
NAVGDRTGA

SEQ ID NO: 94 - protein, 14 aa, Artificial Sequence
  source: /note=“Description of Artificial Sequence Synthetic peptide”
AAGLVSLRTHTWEY

SEQ ID NO: 95 - protein, 21 aa, Artificial Sequence
  source: /note=“Description of Artificial Sequence Synthetic peptide”
AAEVGYGYQGPPLTTPSMYDY

SEQ ID NO: 96 - protein, 14 aa, Artificial Sequence
  source: /note=“Description of Artificial Sequence Synthetic peptide”
AAGLLSLRSHTWEL

SEQ ID NO: 97 - protein, 14 aa, Artificial Sequence
  source: /note=“Description of Artificial Sequence Synthetic peptide”
AAGLLSLRTHTWEY

SEQ ID NO: 98 - protein, 19 aa, Artificial Sequence
  source: /note=“Description of Artificial Sequence Synthetic peptide”
AVKSQTYSTDYVQPRRYAY

SEQ ID NO: 99 - protein, 14 aa, Artificial Sequence
  source: /note=“Description of Artificial Sequence Synthetic peptide”
NAASLAIVRGINNY

SEQ ID NO: 100 - protein, 14 aa, Artificial Sequence
  source: /note=“Description of Artificial Sequence Synthetic peptide”
AAGLVSLLTHTWEY

SEQ ID NO: 101 - protein, 724 aa, Eimeria maxima
MALLPTQRLAPGWALSLLVFLAAGLTFHSSHAAASSEADQVCTRLLDVMLVVDESGSIGTSNYGKVRSFISNFAGTMPLSPDDVRVGLVTFGTSAVTRWDLSDSRAQNADLLAAAAKKLPYAAGSTYTHLGLAKAEEILFSFQKGGRDNAPKMILVMTDGASSRRSQTLSAAEKLRNRGVIIVVLGVGTGVNSAECRSIAGCDTSDTVECPRYLQSNWGGVSSQINGIIKAACKDLAKDAVCSEWSEYGPCEGECGTEGTRTSTRVEIAPPRPGTPPCPTCEAPQGRSCAQQPPGLMRTEQCTMPACKIDAHCGDFGPWSEWSTTCGSATRQRVRQGYEDPPASGGGLSCIDQNPPKYAKEVEVVQKSPCPVQQQPGPWSDWSDCSATCGGGTRYREREGYPQEGELFGGQTLQAQGLDVRETDTCNENPCPVDATCGEWTEFSDCSRVCGGGTKERRREPWLDNAQFGGRSCSQQHPEGPTESVECNEHPCPVDEVVGEWEDWGPCSEQCGRGRQFRYRGPSLQQAMFGGKTIEEQNAGVPEEQKILKVEERPCNDVPCGPCTLPFTEWTACESCSGTRTRDSMVAFDYDDRQCQNPTHEEESCDAVCEESASGGGVGGGAGGAGGGGGGSAGGEGSNGAGPGEDKEEESKGFPTAAVAGGVAGGVLAIAAGAGAFYGLSGGAASAAGGAAAEVMVESGTANPPEVEKESLITAGEQSEMWAS

SEQ ID NO: 102 - protein, 712 aa, Eimeria tenella
MAPLPRRRLAPCRALSLLVGLLAASFAFSSLQPGATTSSGQDQVCTSLLDVMLVVDESGSIGTSNFRKVRQFIEDFVNSMPIPPEDVRVGLITFATRSKVRWNLSDPKATNPSLAISAARSLSYSTGVTYTHYGLQDAKKLLYDTNAGARNNVPKLVLVMTDGASNLPSQTRSSAAALRDAGAIVVVLGVGSGVNSSECRSIAGCSTSNCPRYLQSNWSNVTQQVNGIIKAACKDLAKDAVCSEWSEYGPCVGECGKEGVQTSTRVEISPQKPGSPPCPTCEAPRGRSCAEQPPGLTRTQPCTMPVCKTDAHCGEFGAWSEWSTTCGTATRKRQREGYNSPPAAGGGLSCMEQNPPKHEFEVETVQKSPCPVQQQPGPWSEWTECSATCGGGTKHREREGLPQEGELYGGQTLEQQGIAVRETASCSENPCPIGATCGEWTEYSACSRTCGGGTQERKREPWLDNAQHGGRTCMEQYPDGPISVRECNTQPCPVDEVVGDWEDWGQCSEQCGGGKRTRNRGPSKQEAMFGGKTVAQQNVELPEGEKIEVVQEGGCNEVPCGPCTLPFSEWTECESCSGHRTRESAVAFDYTDRMCSGDTHEVHSCEEYCSQNAGGGAGGDGGAGGGTGGSGEEEGKEESSGFPTAAVAGGVAGGVLAIAAGAGAFYGLSGGSAAAATEAGAEVMTEAGTSNAAEVEKESLISAGEQSEMWAS

SEQ ID NO: 103 - protein, 295 aa, Eimeria maxima
MARALSLIALGLLFSLPSTSAVRTKVPGDEPSGPDSALTAGSPTRGNHGVGGFAEAHCNRLTVRGGLQEKEAVKVTANGWGKGDADFFVELVTDNTRGMLQIRESQTEAGPGLAGGGGQEGGSTEGSKTDEGPVKEQPSIPIVGVRIPGSANENGESRKPAVLVYGEGESAPKEFPLDSPAGPTSPFMVVLQQKSPTEMTVRLFTWIPNGSGGDGSWHETFVDVGVGINHRDVMVVVSDCVPHSLRIYGSSSADLVTADEKTCQATEPQLVNLTSPPENRTHPGAETSTTTSVSS

SEQ ID NO: 104 - protein, 342 aa, Eimeria tenella
MARALSLVALGLLFSLPPSSAVRTRVPGEDSFSPESGVLSGTDAPERRPIVPGLVEGNCGRLTVRNGPSVDETIKVTSAGWTKSERDFIVSLVADETRKVVQLRESEGASGASGPGPAPAEKPPSGQGSAEEAPKGEGGQEKPSVPLIAVRIHGSGGDKGESAPQSAVLLYGNDESEPTEVPLETAAGPTTPLMVLITQQNPKEVEVRVLAWISTDATTGKGSWKENSVVVGSSLSGRDLTVNLSDCGPSSLRVYGSASADLVTVKEGMCEADDPELIALTRPHTSAASPLPAEEGDVAQDAQQSAGAQQEAEAQEVGEPQQEAAAAEQGSSAAESDTQQSS

SEQ ID NO: 105 - protein, 539 aa, Eimeria maxima
MCGLRAAFTEAVCLGLLSLGSTVVQGIKDKVHQGHTEAAATAAAGNLSAELHAALHQPNDNPFLVPPLSDFMDRFNIPKVHGSGIYVDLGGDKEVDGRTYREPSGLCPVFGKTIVLYQPQNNPNYKNDFLDDMPTKQQSDAVGHPLPGGFNNSFKMPDKSPYSPMSAQKLNSYKQLKANTPMGKCAEMSFMTTAGKNSSYRYPWVYDTKRDLCYFLYLPVQRLMGERYCSVDGKPDGMTWYCFEPHKALDSRPELVYGSAYVGRDPDYWETHCPNKAVKDAVFGVWVSGRCTEHKHLDGAKKEKVNSKAECWSLAFENPEVASDHPVTEDENFGTYGYFFPSTEPNQPKSGGEGVNFASFYPGSMECWLSGEIPTCLVPLEGAAAFTALGSLEEETAPCTDSFPQTKTPCDRNTCTQIVATCVSGTLVSEEVPCSPEDGTRCEGGFPKGVMIGLAAAGGILLLLLGGGGFLLYRSRMRPAAKGDEATRSDYVQEEAAANRRKQRQSDLVQQAEPSFWEEAEADEAETGESTHVLVDQDY

SEQ ID NO: 106 - protein, 536 aa, Eimeria tenella
MRRLSPALGLLAAALSCAGPAAGVQHKLQHRQQQQQQHSHASTSHAAAVLAASSDASTDSNPFMQPPYAEFMARFNIPKVHGSGVYVDLGNDKEVKGKMYREPGGRCPVFGKNIEFYQPLDSDLYKNDFLENVPTEEAAAAAKPLPGGFNNNFLMKDKKPFSPMSVAQLNSYPQLKARTGLGKCAEMSYLTTAAGSSYRYPFVFDSKKDLCYLLLVPLQRLMGERYCSTRGSPPGLSHFCFKPLKSVSLRPHLVYGSAYVGERPDDWETKCPNKAVKDAVFGVWEGGRCEEQRLRLGAQTAAAAAKEDCWALAFNNPFAASDQPTSQDEAATSPGYYFPSITPSQPKSGGVGVNFASYYPSGECVLSGEVPTCLLPRQGAAAFTSVGSLEEEELPHCDPTFPASLGSCDPSSCKAILTECRGGRLVEQQTDCVPEDGSKCESKGGGVFIGLAVAGGLLLLLLTGGAFFIYKQRQKALPKESSPQRTDFVQDEAATGRGKKRQSDLVQQAEPSFWEEAEADEPHADENTQVLLDQEY

SEQ ID NO: 107 - protein, 1124 aa, Eimeria maxima
MFRLLYLPGLVTILSVSQRTPEVKITMLGAFSIFALSTLLSLPPYSWRMAAMADSLSVTPEYEVEGPTNFLTPSLVTLDSALEGLDSGGTPGFSGQYADLLDRICPADSPALDLPQQPTREQNIGELELMLSDNDLGEATNKLWLAFYGHEVPKAASSVEELSAQFLELVGQVRVAFQDVHHHMVKQEGHPEFHNSQLPRSIVPIVGARNPLMLGLFWNLLIAYTGFDSYFGEDSITLPFFSWPSLLASLGGQSSSHIDAMCSVQRSRSLTEKFFKWRSPRGIQQNRRHKRVSSLRESSHRTFCELIDRLISSLGEFVAGHVTTLAAAGVPVELGISPLQNMKRLHAETCLPGEDGHILAPCIFEESRLALDSQQLVKMEDDTDQQIRHSAQAELLKQAHTTISGLGALDCKLSSRKWRALPYTPMPRLFKLPELAQLRKDWILERLNLAVATMLTTSSLDNASNYWLAFDRRNFKRAAQDFKKHTIDVLRSVGMEAIGRRLLQSSGSEISDDEVQAAASRLKDLRAVSDGMRVALALYAILNAGRHAQSAHVGSQGHAIHAQCYGTCCSTAEPYAYVGVRVPELSGRDAQEHERNQDPESQKGAWSKIDLNGLPLPDLTDWWSRLNYIVVQALESYLVLEKHLEFLEDTVFTLVSDSLRKLKADAGQTLFFTRIAHQKRQGPLQRLWEGAKKSLMGFLYRSPGRQHGIWFGVTVDFEQLHDLLGQLKLVIEAEPRLTIKINMQEALLREMETRIRVEGSDISRVPPLVERNMGMLGVRRAYGTLSEGLRDTEFQRSMCIDHCRGLWQMALSTMLPSMLSPDIFRKYEKAFGTPWALEHLSDPALVNSRRMVLKSDAALNFFDHSTPKEVREELKGLESGQASMFAYYMLFSSRAHQVLGNHYLGLYLRQQAPFMGNMVVDWISTRRKHAVAAIVSSFVLTFMGIYAVMSFVDILQNLIVSGAPPPFDCLWNPVFGEMACSPVPGGASLGTAWATAIEQVFLIGLFSGTAGGFSLFLTINSAIAVVVNQSKTLMRLQMCLGSAISRLLRRGKRSFSRIREYFIKRSSVKRVMLQRAVAGMKAGSSTATMSNSETLEAADQLLDRLTRTGRANPLKASGGKPAFK

SEQ ID NO: 108 - protein, 37 aa, Eimeria maxima
DILQNLIVSGAPPPFDCLWNPVFGEMACSPVPGGASL

SEQ ID NO: 109 - protein, 1402 aa, Eimeria tenella
MGVFSRLLSAALLCAAASPALLSPQAAAAAAAPAEGSTSFTLPSGGSLHVQVLRGPQQQQQQQQQGPWGSTMSFAGPTGATGGYSVTDSATLRSSAGPPAAAAAAAAAEGVYELPTPPTPENPISKTQVLFGWVPEAAAAASLQAVNAEELNNALRSQAFVRKLRTRALVEKSCMIPLASDRRSYLEAFWSAAAAERKFRAEAESQRAAIEEELKQQQMAAPDMSYARSVATRANIESGASPAAAAAAAAGAAAAAAAGEEAAAAALRYQETVDRVLANRIEVLMTCKLAALLGQPGIFLNDKIAAAEVLDLVATALGIGNTKEEAAAAAASPIVPMFGGLEPFLLASNPLIVGHVLTLLIAHIDREAFFGEAARKAFYSFTSLAAAAGGEGAVGMLDEMCDRDRGPRLAAPLLGKYRPKGRGGKPRKRGSSSPWFVRGASRVHRNKLQPELLRAYCDATEMILNALMLKQEDVQQEMLKFSLPVEPLVDPATNASRIQTKTCRGGAPVCEFENSILSPIANPLDPQVLEQNTNTRAAFNLYAGLASAQLGNLLEETGSKYYDVRADQWLEIISKPTAHSDILEKVFFYDNREFLASKRSKLIKSFETNAKKIAALAGKPAVSISEAAAAYEEIQRSVGSQTGKKGAAAAKKNLFPTAATLLLKSTLACDMTSSFAAKLESFTQFMFKAKAASGRRQAPLQRTLMAFIRTGKGSALQSMCSYDPLTFNYLFLYRFVLLGSDPAKTHDKQHAAVSKTPLTTRLLGSTWTPSILKRVLRGVKRKSSILKAKQLLLESLDAGVFPLLLTSFEWIVHTQAALQVNQNSEMFHELEAQQPRALPAEPQKAAEAKLHEGGLVKRTDEQLAEWAEFGVPAALREQLRRGEKLPKAVALERIPTPDLTQWEQQLNRKWLAALEAYLRHPYGAAAAAARDPVALLVQRSRDRLEAELDSTIFLGRIVGAPRGPRRGRRALRAVGGLFRALLRAPRQSEFAVWMGVKVHADRVLRVLHAVHAAAEVAKTAGLFEHVREAFLELVRDAVLGSLQNQLRVPGFDTFAAVEAGLRGGEVSAAAAAAAAAAAAARRSAGFLSVHPELAALGPAAREAEFQNSMCMDHCEALWTLVTSLVFSALQNPRKWRDYEKEVGGAAAAAALADPRRVNSFRLGLSVQTDFFENVLDKKSKRNIQKMKFGGGSWFAYALLLAARLHRGLGHSDLATFFSFQAPYLGHFVLQWQQQRREARRKALLSMLSLGFFFAYTFISVSDITQHLNDSGLGPAVECLENLVVGPVCPAAVVAPAVRSAAAAAAADVFKVGLFGLLTPYLVWPMAAAAAWQLLRSEFKVLLQFEMSLKSLFSRFSSWVRRPFARWWQQRRRLKQLLLQSAAAKFRTEKKRLHGRDPPPRDF

SEQ ID NO: 110 - protein, 37 aa, Eimeria tenella
DITQHLNDSGLGPAVECLENLVVGPVCPAAVVAPAVR

SEQ ID NO: 111 - protein, 121 aa, Artificial Sequence
  source: /note=“Description of Artificial Sequence Synthetic polypeptide”
QVQLQESGGGLVQAGGSLRLSCKISGNTFSSYTMGWYRRPPGKQRELVAQLTKGGSTNYADSVKDRFTISTDNAKNTVYLQMDSLKPEDTAVYYCNMKTAWTIGTRGYDYWGQGSQVTVSS

SEQ ID NO: 112 - protein, 121 aa, Artificial Sequence
  source: /note=“Description of Artificial Sequence Synthetic polypeptide”
QVQLQESGGGLVQAGGSLRLSCAISRSTFSSYTMGWYRRPPGKERELVAQITNGGSTNYADSVKGRFTISRDNAKNTVYLQMNSLKPEDTAVYYCNMKTAWTIGTRGYDYWGQGSQVTVSS

SEQ ID NO: 113 - protein, 120 aa, Artificial Sequence
  source: /note=“Description of Artificial Sequence Synthetic polypeptide”
QVQLQESGGGLVQAGGSLRLSCVASGSSISSYAMAWYRQAPGQQRELVAGISSGGATEYADSAKARFTISRDNAKNTVYLQLYNLKPEDTAVYYCNMRQLPRWSQLLGSWGQGTQVSVSS

SEQ ID NO: 114 - protein, 120 aa, Artificial Sequence
  source: /note=“Description of Artificial Sequence Synthetic polypeptide”
QVQLQQSGGGLVQAGGSLRLSCSASGSTISSYAMAWYRQAPGQQRELVAGISSGGGTQYADSAKGRFTISRDNAKNTVYLQLHTLKPEDTAVYYCNMRQLPRWSLLLGAWGQGTQVSVSS

SEQ ID NO: 115 - protein, 113 aa, Artificial Sequence
  source: /note=“Description of Artificial Sequence Synthetic polypeptide”
QVQLQQSGGGLVQAGGSLRLSCAASGSTLTRYSVSWYRQAPGNEREVVSRILKGGSTHYADSVKGRFTISRDNAKNTVSLQMNSLKPEDTAVYYCHLDWTDFWGQGTQVTVSS

SEQ ID NO: 116 - protein, 120 aa, Artificial Sequence
  source: /note=“Description of Artificial Sequence Synthetic polypeptide”
QVQLQQSGGGLVQAGGSLRLSCAASGSSFSSYAMGWYRQAPGKQREWVAGIGSLGSPNYADSVKGRFVMSRDNAKNTVYLQMNSLKPEDTAVYYCNMRLLTTWYNLLGSWGQGTQVTVSS

SEQ ID NO: 117 - protein, 117 aa, Artificial Sequence
  source: /note=“Description of Artificial Sequence Synthetic polypeptide”
QVQLQESGGGLVEAGGSLRLSCVASRNIFGVAHMTWYRQAPGKERELVATVSSSGTTNHVDSVKGRFTISRDNSKTALYLQMNTLKPEDTAVYVCRIATNVPPYNYWGQGIQVTVSS

SEQ ID NO: 118 - protein, 120 aa, Artificial Sequence
  source: /note=“Description of Artificial Sequence Synthetic polypeptide”
QVQLQESGGGLVQPGGSLRLSCAASGSSISNYAMAWYRQAPGKQREWVAGISQRMDTIYADSVKGRFTISRDNAKNTVSLQMNSLKPEDTAVYYCNIRILPTWATTVGSWGQGTQVTVSS

SEQ ID NO: 119 - protein, 124 aa, Artificial Sequence
  source: /note=“Description of Artificial Sequence Synthetic polypeptide”
QVQLQESGPGLVKPSQTLSLTCTVSGGSITTNRYYWSWIRQPPGKGLEWMGAIAYDGSTYYSPSLKSRTAISRDTSKNQFSLQLSSVTPEDTAVYYCTRGGDYSSNDYYGMDYWGKGTLVTVSS

SEQ ID NO: 120 - protein, 113 aa, Artificial Sequence
  source: /note=“Description of Artificial Sequence Synthetic polypeptide”
QVQLQESGGGLVQAGGSLRLSCAASGSSLSRYSVSWYRQAPGDEREVVSRLTSRGDNFYADSVKGRFTISRDNAKNTVYLQMNSLKPEDTAVYYCHLDWTDFWGQGTQVTVSS

SEQ ID NO: 121 - protein, 120 aa, Artificial Sequence
  source: /note=“Description of Artificial Sequence Synthetic polypeptide”
QVQLQESGGGLVQAGGSLRLSCVASGSDVSTYALGWYRQAPGKQREWVAGISRGGDTNYADSVKGRFTISRDYAKNTVYLQMNSLKPEDTAVYYCNIRILPNWATTVGRWGQGTQVTVSS

SEQ ID NO: 122 - protein, 120 aa, Artificial Sequence
  source: /note=“Description of Artificial Sequence Synthetic polypeptide”
QVQLQESGGGLVQAGGSLRLSCVASGSSGSDYALGWYRQAPGKQREWVAGISRGGDTNYADSVQGRFTISRDNAKNTVYLQMNSLKPEDTAVYYCNMRILPNWNPIVGRWGQGTQVTVSS

SEQ ID NO: 123 - protein, 120 aa, Artificial Sequence
  source: /note=“Description of Artificial Sequence Synthetic polypeptide”
QVQLQESGGGLVQAGGSLRLSCAAPVRTFSNYAMGWYRQAPGKQRELVAGISSNGRTDYVDSVKGRFTISRDNAKNTVSLQMNSLKPEDTAVYYCNMRLTTSWERLLGAWGQGTQVTVSS

SEQ ID NO: 124 - protein, 123 aa, Artificial Sequence
  source: /note=“Description of Artificial Sequence Synthetic polypeptide”
QVQLQESGGGLVQAGGSLRLSCSASGSTFSTFSRYAMAWYRQAPGQQREWVAGISSGGSTTYADSAKGRFTISRDNAKNTVYLQLHSLKPEDTAVYYCNMRQLPSWSQLLGSWGQGTQVSVSS

SEQ ID NO: 125 - protein, 123 aa, Artificial Sequence
  source: /note=“Description of Artificial Sequence Synthetic polypeptide”
QVQLQESGGGLVQTGGSLRLACAASGDSFILYTAGWYRQAPGKERELVAQITSGGTTNYGDFAKGRFTIFRDDAKNMVILQMASLKPEDTAVYYCNAKKVRSWPITEEPRDYWGQGTQVTVSS

SEQ ID NO: 126 - protein, 113 aa, Artificial Sequence
  source: /note=“Description of Artificial Sequence Synthetic polypeptide”
QVQLQESGGGLVQPGGSLRLSCAASGSTLTRYSVSWYRQAPGNERDVVARILKGGSTHYSDSVKGRFTISRDNAKNTVSLQMNSLGPEDTAVYYCHLDWTDFWGPGTQVTVSS

SEQ ID NO: 127 - protein, 121 aa, Artificial Sequence
  source: /note=“Description of Artificial Sequence Synthetic polypeptide”
QVQLQESGGGLVQAGGSLRLSCAASGSSISSYAMGWYRQAPGKQREWVAVIGSGGTTWYADSVKGRSTISRDNAKNTVYLQMNSLKPEDTAVYYCNMRLSTLSLHRALGAWGQGTRVTVSS

SEQ ID NO: 128 - protein, 127 aa, Artificial Sequence
  source: /note=“Description of Artificial Sequence Synthetic polypeptide”
QVQLQESGGGLVQAGGSLRLSCSASGSTFTSSGSTFSRYAMAWYRQATGQQRELVAGISSGGSTQYADFARGRFTISRDNAKDTVYLQMNNLQPEDTAVYYCNIRILPRWDQTVGSWGQGTQVTVSS

SEQ ID NO: 129 - protein, 126 aa, Artificial Sequence
  source: /note=“Description of Artificial Sequence Synthetic polypeptide”
QVQLQESGGGLVQAGGSLRLSCVASGSIFTAGSTFNTYAMAWYRQAPGQSRELVAGISSSGSTEYADSVKGRFTISRDNAKNTVYLQLHSLKPEDTAVYYCNMRQLPQWSTLLGAWGQGTQVSVSS

SEQ ID NO: 130 - protein, 116 aa, Artificial Sequence
  source: /note=“Description of Artificial Sequence Synthetic polypeptide”
QVQLQQSGGGLVQAGGSLTLSCAISGNTYNAHTMAWYRQAPGKQRELVARMDFNGESNYDNSVKGRFTISRDNAENTLFLQMNSLRPDDTAVYYCKGSWYFLGDYWGQGTQVTVSS

SEQ ID NO: 131 - protein, 116 aa, Artificial Sequence
  source: /note=“Description of Artificial Sequence Synthetic polypeptide”
QVQLQESGGGTVQAGGSLRLSCAVSGSTFSSYSMNWYRQGPGNQREWVGGIRSNDSTYYADSVKGRFTISRDNAKNAWNLQMNSLKSEDTAVYYCNFWDGSFNQYWGQGTQVTVSA

SEQ ID NO: 132 - protein, 117 aa, Artificial Sequence
  source: /note=“Description of Artificial Sequence Synthetic polypeptide”
QVQLQESGGGVVQAGGSLRLSCVASGSDTSVYAMRWWRQVPGKERLLLASITSGGTVRYAESVKGRFTISRDDAKNTLYLQMNSLKTEDTAVYLCNAERVLARQNYWGQGTQVTVSS

SEQ ID NO: 133 - protein, 121 aa, Artificial Sequence
  source: /note=“Description of Artificial Sequence Synthetic polypeptide”
QVQLQESGGGLVQAGGSLRLSCAASGSIFSINVIGWYRQVPGKERELVANSGSTTKYADSVKGRFTISRDNAKNTVDLQMNSLKPEDTAVYYCNAVVTSRTSLGLRSYVYWGQGTQVTVSS

SEQ ID NO: 134 - protein, 120 aa, Artificial Sequence
  source: /note=“Description of Artificial Sequence Synthetic polypeptide”
QVQLQESGGGLVQAGGSLRLSCVALGNIGWINDMGWYRQAPGKERELVARISSAEATNYADAVKGRFTISRDDAKNTVYLQMNSLKPEDTAVYSCHTRRWYDDGYDYDTWGQGTQVTVSS

SEQ ID NO: 135 - protein, 121 aa, Artificial Sequence
  source: /note=“Description of Artificial Sequence Synthetic polypeptide”
QVQLQQSGGGLVQAGGSLRLSCAASGSIFSINVIGWYRQVPGKERELVAKSDRTTKYADSVKGRFTISRDNAKNTVDLQMNSLKPEDTAVYYCNAVVTSRTSQGLRSYAYWGQGTQVTVSS

SEQ ID NO: 136 - protein, 112 aa, Artificial Sequence
  source: /note=“Description of Artificial Sequence Synthetic polypeptide”
QVQLQQSGGGLVQSGGSLRLSCTATRSTISGYGGRWYRQAPGKQREFVALLSSGGMTRYADSVMGRFTISRDDIKNTLYLQMNTLKPEDTAVYYCNTFDGAWGQGTQVTVSS

SEQ ID NO: 137 - protein, 121 aa, Artificial Sequence
  source: /note=“Description of Artificial Sequence Synthetic polypeptide”
QVQLQQSGGGLVQAGGSLRLSCTTSGIIFSFSLLNVGWYRQAPGKERELVARMSTAGNTYYSTSVKDRFTISRDNGKNTVDLQMNSLNADDTAVYYCNLRRSWAPSDSGFWGQGTQVFVSS

SEQ ID NO: 138 - protein, 118 aa, Artificial Sequence
  source: /note=“Description of Artificial Sequence Synthetic polypeptide”
QVQLQESGGGMVQAGGSLRLSCAASGTTITINTMRWYRQPPGKERELVASIVPSGKTYYADSVKGRFTISRDNHKNTMYLQMNSLKPEDTAVYYCNSDGRFRGIGTYWGQGTQVTVSS

SEQ ID NO: 139 - protein, 121 aa, Artificial Sequence
  source: /note=“Description of Artificial Sequence Synthetic polypeptide”
QVQLQESGGGLVQAGGSLRLSCAASGSIFSINVIGWYRQAPGKERELVANSGSTTKYADSVKGRFTISRDNAKNTVDLQMNSLKPEDTAVYYCNAVVTSRTSLGLRSYAYWGQGTQVTVSS

SEQ ID NO: 140 - protein, 121 aa, Artificial Sequence
  source: /note=“Description of Artificial Sequence Synthetic polypeptide”
QVQLQESGGGLVQAGGSLRLSCVASGITFNFNYYVMGWYRQAPGKQRELVARISDGGNTNYADSVKGRFTISRDNTKNTGYLQMNSLKPEDTAIYYCNLRRSWAPADNGHWGQGTQVTVSS

SEQ ID NO: 141 - protein, 118 aa, Artificial Sequence
  source: /note=“Description of Artificial Sequence Synthetic polypeptide”
QVQLQESGGGLVQAGGSLRLSCAASGIAFSMFDMVWYRQAPGKQRELVASVSDGGSTAYADSVKGRFTISRDNAKKMVYLQMNSLKPEDTAVYYCKAARSWAYGSGYWGQGIQVTVSS

SEQ ID NO: 142 - protein, 115 aa, Artificial Sequence
  source: /note=“Description of Artificial Sequence Synthetic polypeptide”
QVQLQQSGGGLVQAGGSLRLSCAASGITPSRHPMTWYRQVPGKKRELVASLTDADSTYYTDSVKGRFTILRDSAANTVYLQLNGLKPEDTAVYYCFAILGGRSYWGQGTQVTVSS

SEQ ID NO: 143 - protein, 116 aa, Artificial Sequence
  source: /note=“Description of Artificial Sequence Synthetic polypeptide”
QVQLQESGGGLVQAGGSLRLSCAASQSITSTYGMAWYRQAPGKQREWVATINHTGNTNYVASVKGRFAISRDNSDYTVYLQMSDLKPEDTAVYYCSPAVWTGLRPWGQGTQVTVSS

SEQ ID NO: 144 - protein, 115 aa, Artificial Sequence
  source: /note=“Description of Artificial Sequence Synthetic polypeptide”
QVQLQESGGGLVQAGGSLRLSCAASGSTPSRHPMTWYRQAPGKSRELLASITNVDSTYYADSVKGRATIFRDNAASTVYLQLNGLKLEDTAVYSCFAVLDGRSYWGQGTQVTVSS

SEQ ID NO: 145 - protein, 116 aa, Artificial Sequence
  source: /note=“Description of Artificial Sequence Synthetic polypeptide”
QVQLQESGGGLVNPGGSLRLSCAASVSTTSTYGMTWYRQAPGKQREWVATINHTGGTNYADSVKGRVAISIDNAKNTVYLQMSDLKPEDTAVYYCSPPVWTGLRPWGQGTQVTVSS

SEQ ID NO: 146 - protein, 114 aa, Artificial Sequence
  source: /note=“Description of Artificial Sequence Synthetic polypeptide”
QVQLQESGGGTVQAGESLTLSCAASGTIFRFTVMGWYRQVPGKEREFVASITYTDATDYADSVKGRFTISRDNAKNTAYLQMNSLKPEDTAVYSCFAHYAGYYYGQGTQVTVSP

SEQ ID NO: 147 - protein, 116 aa, Artificial Sequence
  source: /note=“Description of Artificial Sequence Synthetic polypeptide”
QVQLQQSGGGLVQAGGSLRLSCTASGITSSTYAMSWYRQAPGKEREPVASMASSGSTFYADSVKGRFTISRDNAKNMVYLQMNSLKPGDTAVYYCKVPRFGGSDYWGQGTQVTVSS

SEQ ID NO: 148 - protein, 116 aa, Artificial Sequence
  source: /note=“Description of Artificial Sequence Synthetic polypeptide”
QVQLQQSGGGLVQPGGSLRLSCTASGITSSTYAMSWYRQAPGKQREPVASIASSGSTFYAESVKGRFTISRDNAKNMAFLQMNSLEPGDTAVYYCKVPRYGGSDYWGQGTQVTVSS

SEQ ID NO: 149 - protein, 116 aa, Artificial Sequence
  source: /note=“Description of Artificial Sequence Synthetic polypeptide”
QVQLQESGGGLVQAGGSLRLSCAASRSSLSTYAMGWYRQAPGKQRELIATITTGGVTQYVDFVKGRFTISRDNAKNTVYLQMNSLKPEDTAVYYCVRSPRTSWTSWGQGTQVTVSS

SEQ ID NO: 150 - protein, 116 aa, Artificial Sequence
  source: /note=“Description of Artificial Sequence Synthetic polypeptide”
QVQLQQSGGGLVQAGGSLRLSCVASASALRMGWYRQAPGEQRELVATLDNAGKTNYAASVKGRFTISRDSAKNTVYLQMNSLKPEDTAVYYCQAHRWTFDGWQDYWGQGTQVTVSS

SEQ ID NO: 151 - protein, 116 aa, Artificial Sequence
  source: /note=“Description of Artificial Sequence Synthetic polypeptide”
QVQLQESGGGLVQAGGSLRLSCTASRSTLSTYAMGWYRQAPGEQRELVATITTGHITQYADFVKGRFTISRDNTKNTVYLQMNSLKPEDTAVYYCVRSPRTSWTSWGQGTQVTVSS

SEQ ID NO: 152 - protein, 116 aa, Artificial Sequence
  source: /note=“Description of Artificial Sequence Synthetic polypeptide”
QVQLQESGGGLVQAGGSLRLSCAASASALRMGWYRQAPGESRELVATIDNAGKTNYADSVKGRFTIAKDSAKNTVYLQMNSLKPGDTAVYYCQAHRWTFDGWQDYWGQGTQVTVSS

SEQ ID NO: 153 - protein, 116 aa, Artificial Sequence
  source: /note=“Description of Artificial Sequence Synthetic polypeptide”
QVQLQESGGGLVQAGGSLRLSCASSASALRMGWYRQAPGEQRELVATMDNAGSTNYAGSVKGRFTISRDSAKNTVYLQMNSLKPGDTAVYYCQARRWTFDGWQDYWGQGTQVTVSS

SEQ ID NO: 154 - protein, 116 aa, Artificial Sequence
  source: /note=“Description of Artificial Sequence Synthetic polypeptide”
QVQLQQSGGGLVQAGGSLRLSCAASGSTLSTYAMGWYRQAPGKQRELVATITTGRVTQYADFVKGRFTISRDNAKNTVYLQMNSLKPEDTAVYYCVRSPRTPWSSWGQGTQVTVSS

SEQ ID NO: 155 - protein, 115 aa, Artificial Sequence
  source: /note=“Description of Artificial Sequence Synthetic polypeptide”
QVQLQQSGGGLVQAGGSLRLSCATRSSLSTYAMGWYRQAPGKQRELVATITTGHITQYADFVKGRFTISRDNAKNMVYLQMNSLKPEDTAVYYCARSPRTSWTVWGQGTQVTVSS

SEQ ID NO: 156 - protein, 127 aa, Artificial Sequence
  source: /note=“Description of Artificial Sequence Synthetic polypeptide”
QVQLQESGGGLVQAGGSLRLSCAASGFSLASYHITWFRQAPGKEREGVSCIGYNSGWTDYGEFVKGRFTISRDNDKNTVYLQMNNLQPEDSAVYYCAARWSFGGQCSYGTHNVQRYRGQGTQVTVSS

SEQ ID NO: 157 - protein, 116 aa, Artificial Sequence
  source: /note=“Description of Artificial Sequence Synthetic polypeptide”
QVQLQQSGGGKVQPGGSLRLSCVASRPVFSMAWYRQAPGKQRVMVASTTNGKEPNYEDSVQGRFTISRDNAKNAVYLQMNSLKPEDTAIYSCKARHWEFDGVKEYWGQGTQVTVSS

SEQ ID NO: 158 - protein, 122 aa, Artificial Sequence
  source: /note=“Description of Artificial Sequence Synthetic polypeptide”
QVQLQESGGGLGQAGGSLRLSCEASGRAFSTYHMGWYRQAPGKQRELVATITSSGNINYADSVKGRFTISRDNAKNTVNLQMNNLKPDDTAVYYCNRGVLSPSDVYWPSTTWGQGTQVTVSS

SEQ ID NO: 159 - protein, 114 aa, Artificial Sequence
  source: /note=“Description of Artificial Sequence Synthetic polypeptide”
QVQLQESGGGTVQAGESLTLSCAASGSIFRFTVMGWYRQVPGKEREFVASITYPGGTEYVDSVKGRFTISRDNAKNTAYLQVSSLKPEDTAVYYCFAHYGSYYYGQGTQVTVSP

SEQ ID NO: 160 - protein, 113 aa, Artificial Sequence
  source: /note=“Description of Artificial Sequence Synthetic polypeptide”
QVQLQESGGGLVQAGGSLRLSCTGPGSTFSSYAVGWYRQAPGGNREWVASISSSGEITRYADSVTGRFTISRDNAKNTVDLQMNSLRPEDTAVYYCAIGTMARGKGTLVTVSS

SEQ ID NO: 161 - protein, 115 aa, Artificial Sequence
  source: /note=“Description of Artificial Sequence Synthetic polypeptide”
QVQLQQSGGGLVQAGGSLRLSCAGSGRPFGSYVMGWYRQAPGEQREMVARMTSAGSGGVADYGESVKGRFAISRDYAKNMVFLQMNSLKPEDTAVYYCWSALGYWGQGTQVTVSS

SEQ ID NO: 162 - protein, 116 aa, Artificial Sequence
  source: /note=“Description of Artificial Sequence Synthetic polypeptide”
QVQLQQSGGGLVQAGGSLRLSCAASRSALSMGWYRQAPGEQRELVATKDNAGVTTYADSVKGRFTVSRDSAKNTVYLQMNSLKPEDTAVYYCQARRWTLDGWQDYWGQGTQVTVSS

SEQ ID NO: 163 - protein, 116 aa, Artificial Sequence
  source: /note=“Description of Artificial Sequence Synthetic polypeptide”
QVQLQESGGGLVQAGGSLRLSCAASASALRMGWYRQAPGEQRELVATIDSAGNTNYAGSVKGRFTISRDSAKNTVYLQMNSLKPGDTAVYYCQARRWTFDGWQDYWGQGTQVTVSS

SEQ ID NO: 164 - protein, 116 aa, Artificial Sequence
  source: /note=“Description of Artificial Sequence Synthetic polypeptide”
QVQLQESGGGLVQAGGSLRLSCTASRSTLSTYAMGWYRQAPGKQRELVATITTGRITQYADFVKGRFTISRDNDKNTVYLQMNSLKPEDTAVYYCARSPRTSWILWGQGTQVTVSS

SEQ ID NO: 165 - protein, 126 aa, Artificial Sequence
  source: /note=“Description of Artificial Sequence Synthetic polypeptide”
QVQLQESGPGLVKPSQTLSLTCTVSGGSITTSYSTWSWIRQPPGKGLEWMGVIADDGSAEYSPSLKSRTSISLDTSKNQFSLQLSSVTPEDTAVYYCARRGGAWGSNWWAYHMDDWGKGTLVTVSS

SEQ ID NO: 166 - protein, 121 aa, Artificial Sequence
  source: /note=“Description of Artificial Sequence Synthetic polypeptide”
QVQLQESGPSLVRPSQTLTLTCTLSGGSITDDHYYFTWIRQLPGKELEWLGTIAAAGNIFPSPSFESRTSISRDTSSNQFTLRLNSATPEDTAVYYCARYLKLGLSGMDYWGKGILVTVSS

SEQ ID NO: 167 - protein, 117 aa, Artificial Sequence
  source: /note=“Description of Artificial Sequence Synthetic polypeptide”
QVQLQESGGGLVQPGGSLRVSCVASGFTFSAAYMSWVRQAPGKGLEWVSTIYSDGTRTYYADSVKGRFTISRDNTKNTVYLEMNSLKPEDTALYYCSRDNFGLGDYWGQGTQVTVSS

SEQ ID NO: 168 - protein, 9 aa, Artificial Sequence
  source: /note=“Description of Artificial Sequence Synthetic peptide”
GNTFSSYTM

SEQ ID NO: 169 - protein, 9 aa, Artificial Sequence
  source: /note=“Description of Artificial Sequence Synthetic peptide”
RSTFSSYTM

SEQ ID NO: 170 - protein, 9 aa, Artificial Sequence
  source: /note=“Description of Artificial Sequence Synthetic peptide”
GSSISSYAM

SEQ ID NO: 171 - protein, 9 aa, Artificial Sequence
  source: /note=“Description of Artificial Sequence Synthetic peptide”
GSTISSYAM

SEQ ID NO: 172 - protein, 9 aa, Artificial Sequence
  source: /note=“Description of Artificial Sequence Synthetic peptide”
GSTLTRYSV

SEQ ID NO: 173 - protein, 9 aa, Artificial Sequence
  source: /note=“Description of Artificial Sequence Synthetic peptide”
GSSFSSYAM

SEQ ID NO: 174 - protein, 9 aa, Artificial Sequence
  source: /note=“Description of Artificial Sequence Synthetic peptide”
RNIFGVAHM

SEQ ID NO: 175 - protein, 9 aa, Artificial Sequence
  source: /note=“Description of Artificial Sequence Synthetic peptide”
GSSISNYAM

SEQ ID NO: 176 - protein, 11 aa, Artificial Sequence
  source: /note=“Description of Artificial Sequence Synthetic peptide”
GGSITTNRYYW

SEQ ID NO: 177 - protein, 9 aa, Artificial Sequence
  source: /note=“Description of Artificial Sequence Synthetic peptide”
GSSLSRYSV

SEQ ID NO: 178 - protein, 9 aa, Artificial Sequence
  source: /note=“Description of Artificial Sequence Synthetic peptide”
GSDVSTYAL

SEQ ID NO: 179 - protein, 9 aa, Artificial Sequence
  source: /note=“Description of Artificial Sequence Synthetic peptide”
GSSGSDYAL

SEQ ID NO: 180 - protein, 9 aa, Artificial Sequence
  source: /note=“Description of Artificial Sequence Synthetic peptide”
VRTFSNYAM

SEQ ID NO: 181 - protein, 12 aa, Artificial Sequence
  source: /note=“Description of Artificial Sequence Synthetic peptide”
GSTFSTFSRYAM

SEQ ID NO: 182 - protein, 9 aa, Artificial Sequence
  source: /note=“Description of Artificial Sequence Synthetic peptide”
GDSFILYTA

SEQ ID NO: 183 - protein, 9 aa, Artificial Sequence
  source: /note=“Description of Artificial Sequence Synthetic peptide”
GSTLTRYSV

SEQ ID NO: 184 - protein, 9 aa, Artificial Sequence
  source: /note=“Description of Artificial Sequence Synthetic peptide”
GSSISSYAM

SEQ ID NO: 185 - protein, 16 aa, Artificial Sequence
  source: /note=“Description of Artificial Sequence Synthetic peptide”
GSTFTSSGSTFSRYAM

SEQ ID NO: 186 - protein, 15 aa, Artificial Sequence
  source: /note=“Description of Artificial Sequence Synthetic peptide”
GSIFTAGSTFNTYAM

SEQ ID NO: 187 - protein, 9 aa, Artificial Sequence
  source: /note=“Description of Artificial Sequence Synthetic peptide”
GNTYNAHTM

SEQ ID NO: 188 - protein, 9 aa, Artificial Sequence
  source: /note=“Description of Artificial Sequence Synthetic peptide”
GSTFSSYSM

SEQ ID NO: 189 - protein, 9 aa, Artificial Sequence
  source: /note=“Description of Artificial Sequence Synthetic peptide”
GSDTSVYAM

SEQ ID NO: 190 - protein, 9 aa, Artificial Sequence
  source: /note=“Description of Artificial Sequence Synthetic peptide”
GSIFSINVI

SEQ ID NO: 191 - protein, 9 aa, Artificial Sequence
  source: /note=“Description of Artificial Sequence Synthetic peptide”
GNIGWINDM

SEQ ID NO: 192 - protein, 9 aa, Artificial Sequence
  source: /note=“Description of Artificial Sequence Synthetic peptide”
GSIFSINVI

SEQ ID NO: 193 - protein, 9 aa, Artificial Sequence
  source: /note=“Description of Artificial Sequence Synthetic peptide”
RSTISGYGG

SEQ ID NO: 194 - protein, 11 aa, Artificial Sequence
  source: /note=“Description of Artificial Sequence Synthetic peptide”
GIIFSFSLLNV

SEQ ID NO: 195 - protein, 9 aa, Artificial Sequence
  source: /note=“Description of Artificial Sequence Synthetic peptide”
GTTITINTM

SEQ ID NO: 196 - protein, 9 aa, Artificial Sequence
  source: /note=“Description of Artificial Sequence Synthetic peptide”
GSIFSINVI

SEQ ID NO: 197 - protein, 11 aa, Artificial Sequence
  source: /note=“Description of Artificial Sequence Synthetic peptide”
GITFNFNYYVM

SEQ ID NO: 198 - protein, 9 aa, Artificial Sequence
  source: /note=“Description of Artificial Sequence Synthetic peptide”
GIAFSMFDM

SEQ ID NO: 199 - protein, 9 aa, Artificial Sequence
  source: /note=“Description of Artificial Sequence Synthetic peptide”
GITPSRHPM

SEQ ID NO: 200 - protein, 9 aa, Artificial Sequence
  source: /note=“Description of Artificial Sequence Synthetic peptide”
QSITSTYGM

SEQ ID NO: 201 - protein, 9 aa, Artificial Sequence
  source: /note=“Description of Artificial Sequence Synthetic peptide”
GSTPSRHPM

SEQ ID NO: 202 - protein, 9 aa, Artificial Sequence
  source: /note=“Description of Artificial Sequence Synthetic peptide”
VSTTSTYGM

SEQ ID NO: 203 - protein, 9 aa, Artificial Sequence
  source: /note=“Description of Artificial Sequence Synthetic peptide”
GTIFRFTVM

SEQ ID NO: 204 - protein, 9 aa, Artificial Sequence
  source: /note=“Description of Artificial Sequence Synthetic peptide”
GITSSTYAM

SEQ ID NO: 205 - protein, 9 aa, Artificial Sequence
  source: /note=“Description of Artificial Sequence Synthetic peptide”
GITSSTYAM

SEQ ID NO: 206 - protein, 9 aa, Artificial Sequence
  source: /note=“Description of Artificial Sequence Synthetic peptide”
RSSLSTYAM

SEQ ID NO: 207 - protein, 6 aa, Artificial Sequence
  source: /note=“Description of Artificial Sequence Synthetic peptide”
ASALRM

SEQ ID NO: 208 - protein, 9 aa, Artificial Sequence
  source: /note=“Description of Artificial Sequence Synthetic peptide”
RSTLSTYAM

SEQ ID NO: 209 - protein, 6 aa, Artificial Sequence
  source: /note=“Description of Artificial Sequence Synthetic peptide”
ASALRM

SEQ ID NO: 210 - protein, 6 aa, Artificial Sequence
  source: /note=“Description of Artificial Sequence Synthetic peptide”
ASALRM

SEQ ID NO: 211 - protein, 9 aa, Artificial Sequence
  source: /note=“Description of Artificial Sequence Synthetic peptide”
GSTLSTYAM

SEQ ID NO: 212 - protein, 8 aa, Artificial Sequence
  source: /note=“Description of Artificial Sequence Synthetic peptide”
SSLSTYAM

SEQ ID NO: 213 - protein, 9 aa, Artificial Sequence
  source: /note=“Description of Artificial Sequence Synthetic peptide”
GFSLASYHI

SEQ ID NO: 214 - protein, 6 aa, Artificial Sequence
  source: /note=“Description of Artificial Sequence Synthetic peptide”
RPVFSM

SEQ ID NO: 215 - protein, 9 aa, Artificial Sequence
  source: /note=“Description of Artificial Sequence Synthetic peptide”
GRAFSTYHM

SEQ ID NO: 216 - protein, 9 aa, Artificial Sequence
  source: /note=“Description of Artificial Sequence Synthetic peptide”
GSIFRFTVM

SEQ ID NO: 217 - protein, 9 aa, Artificial Sequence
  source: /note=“Description of Artificial Sequence Synthetic peptide”
GSTFSSYAV

SEQ ID NO: 218 - protein, 9 aa, Artificial Sequence
  source: /note=“Description of Artificial Sequence Synthetic peptide”
GRPFGSYVM

SEQ ID NO: 219 - protein, 6 aa, Artificial Sequence
  source: /note=“Description of Artificial Sequence Synthetic peptide”
RSALSM

SEQ ID NO: 220 - protein, 6 aa, Artificial Sequence
  source: /note=“Description of Artificial Sequence Synthetic peptide”
ASALRM

SEQ ID NO: 221 - protein, 9 aa, Artificial Sequence
  source: /note=“Description of Artificial Sequence Synthetic peptide”
RSTLSTYAM

SEQ ID NO: 222 - protein, 11 aa, Artificial Sequence
  source: /note=“Description of Artificial Sequence Synthetic peptide”
GGSITTSYSTW

SEQ ID NO: 223 - protein, 10 aa, Artificial Sequence
  source: /note=“Description of Artificial Sequence Synthetic peptide”
GSITDDHYYF

SEQ ID NO: 224 - protein, 9 aa, Artificial Sequence
  source: /note=“Description of Artificial Sequence Synthetic peptide”
GFTFSAAYM

SEQ ID NO: 225 - protein, 7 aa, Artificial Sequence
  source: /note=“Description of Artificial Sequence Synthetic peptide”
LTKGGST

SEQ ID NO: 226 - protein, 7 aa, Artificial Sequence
  source: /note=“Description of Artificial Sequence Synthetic peptide”
ITNGGST

SEQ ID NO: 227 - protein, 7 aa, Artificial Sequence
  source: /note=“Description of Artificial Sequence Synthetic peptide”
ISSGGAT

SEQ ID NO: 228 - protein, 7 aa, Artificial Sequence
  source: /note=“Description of Artificial Sequence Synthetic peptide”
ISSGGGT

SEQ ID NO: 229 - protein, 7 aa, Artificial Sequence
  source: /note=“Description of Artificial Sequence Synthetic peptide”
ILKGGST

SEQ ID NO: 230 - protein, 7 aa, Artificial Sequence
  source: /note=“Description of Artificial Sequence Synthetic peptide”
IGSLGSP

SEQ ID NO: 231 - protein, 7 aa, Artificial Sequence
  source: /note=“Description of Artificial Sequence Synthetic peptide”
VSSSGTT

SEQ ID NO: 232 - protein, 7 aa, Artificial Sequence
  source: /note=“Description of Artificial Sequence Synthetic peptide”
ISQRMDT

SEQ ID NO: 233 - protein, 7 aa, Artificial Sequence
  source: /note=“Description of Artificial Sequence Synthetic peptide”
IAYDGST

SEQ ID NO: 234 - protein, 7 aa, Artificial Sequence
  source: /note=“Description of Artificial Sequence Synthetic peptide”
LTSRGDN

SEQ ID NO: 235 - protein, 7 aa, Artificial Sequence
  source: /note=“Description of Artificial Sequence Synthetic peptide”
ISRGGDT

SEQ ID NO: 236 - protein, 7 aa, Artificial Sequence
  source: /note=“Description of Artificial Sequence Synthetic peptide”
ISRGGDT

SEQ ID NO: 237 - protein, 7 aa, Artificial Sequence
  source: /note=“Description of Artificial Sequence Synthetic peptide”
ISSNGRT

SEQ ID NO: 238 - protein, 7 aa, Artificial Sequence
  source: /note=“Description of Artificial Sequence Synthetic peptide”
ISSGGST

SEQ ID NO: 239 - protein, 7 aa, Artificial Sequence
  source: /note=“Description of Artificial Sequence Synthetic peptide”
ITSGGTT

SEQ ID NO: 240 - protein, 7 aa, Artificial Sequence
  source: /note=“Description of Artificial Sequence Synthetic peptide”
ILKGGST

SEQ ID NO: 241 - protein, 7 aa, Artificial Sequence
  source: /note=“Description of Artificial Sequence Synthetic peptide”
IGSGGTT

SEQ ID NO: 242 - protein, 7 aa, Artificial Sequence
  source: /note=“Description of Artificial Sequence Synthetic peptide”
ISSGGST

SEQ ID NO: 243 - protein, 7 aa, Artificial Sequence
  source: /note=“Description of Artificial Sequence Synthetic peptide”
ISSSGST

SEQ ID NO: 244 - protein, 7 aa, Artificial Sequence
  source: /note=“Description of Artificial Sequence Synthetic peptide”
MDFNGES

SEQ ID NO: 245 - protein, 7 aa, Artificial Sequence
  source: /note=“Description of Artificial Sequence Synthetic peptide”
IRSNDST

SEQ ID NO: 246 - protein, 7 aa, Artificial Sequence
  source: /note=“Description of Artificial Sequence Synthetic peptide”
ITSGGTV

SEQ ID NO: 247 - protein, 5 aa, Artificial Sequence
  source: /note=“Description of Artificial Sequence Synthetic peptide”
SGSTT

SEQ ID NO: 248 - protein, 7 aa, Artificial Sequence
  source: /note=“Description of Artificial Sequence Synthetic peptide”
ISSAEAT

SEQ ID NO: 249 - protein, 5 aa, Artificial Sequence
  source: /note=“Description of Artificial Sequence Synthetic peptide”
SDRTT

SEQ ID NO: 250 - protein, 7 aa, Artificial Sequence
  source: /note=“Description of Artificial Sequence Synthetic peptide”
LSSGGMT

SEQ ID NO: 251 - protein, 7 aa, Artificial Sequence
  source: /note=“Description of Artificial Sequence Synthetic peptide”
MSTAGNT

SEQ ID NO: 252 - protein, 7 aa, Artificial Sequence
  source: /note=“Description of Artificial Sequence Synthetic peptide”
IVPSGKT

SEQ ID NO: 253 - protein, 5 aa, Artificial Sequence
  source: /note=“Description of Artificial Sequence Synthetic peptide”
SGSTT

SEQ ID NO: 254 - protein, 7 aa, Artificial Sequence
  source: /note=“Description of Artificial Sequence Synthetic peptide”
ISDGGNT

SEQ ID NO: 255 - protein, 7 aa, Artificial Sequence
  source: /note=“Description of Artificial Sequence Synthetic peptide”
VSDGGST

SEQ ID NO: 256 - protein, 7 aa, Artificial Sequence
  source: /note=“Description of Artificial Sequence Synthetic peptide”
LTDADST

SEQ ID NO: 257 - protein, 7 aa, Artificial Sequence
  source: /note=“Description of Artificial Sequence Synthetic peptide”
INHTGNT

SEQ ID NO: 258 - protein, 7 aa, Artificial Sequence
  source: /note=“Description of Artificial Sequence Synthetic peptide”
ITNVDST

SEQ ID NO: 259 - protein, 7 aa, Artificial Sequence
  source: /note=“Description of Artificial Sequence Synthetic peptide”
INHTGGT

SEQ ID NO: 260 - protein, 7 aa, Artificial Sequence
  source: /note=“Description of Artificial Sequence Synthetic peptide”
ITYTDAT

SEQ ID NO: 261 - protein, 7 aa, Artificial Sequence
  source: /note=“Description of Artificial Sequence Synthetic peptide”
MASSGST

SEQ ID NO: 262 - protein, 7 aa, Artificial Sequence
  source: /note=“Description of Artificial Sequence Synthetic peptide”
IASSGST

SEQ ID NO: 263 - protein, 7 aa, Artificial Sequence
  source: /note=“Description of Artificial Sequence Synthetic peptide”
ITTGGVT

SEQ ID NO: 264 - protein, 7 aa, Artificial Sequence
  source: /note=“Description of Artificial Sequence Synthetic peptide”
LDNAGKT

SEQ ID NO: 265 - protein, 7 aa, Artificial Sequence
  source: /note=“Description of Artificial Sequence Synthetic peptide”
ITTGHIT

SEQ ID NO: 266 - protein, 7 aa, Artificial Sequence
  source: /note=“Description of Artificial Sequence Synthetic peptide”
IDNAGKT

SEQ ID NO: 267 - protein, 7 aa, Artificial Sequence
  source: /note=“Description of Artificial Sequence Synthetic peptide”
MDNAGST

SEQ ID NO: 268 - protein, 7 aa, Artificial Sequence
  source: /note=“Description of Artificial Sequence Synthetic peptide”
ITTGRVT

SEQ ID NO: 269 - protein, 7 aa, Artificial Sequence
  source: /note=“Description of Artificial Sequence Synthetic peptide”
ITTGHIT

SEQ ID NO: 270 - protein, 8 aa, Artificial Sequence
  source: /note=“Description of Artificial Sequence Synthetic peptide”
IGYNSGWT

SEQ ID NO: 271 - protein, 7 aa, Artificial Sequence
  source: /note=“Description of Artificial Sequence Synthetic peptide”
TTNGKEP

SEQ ID NO: 272 - protein, 7 aa, Artificial Sequence
  source: /note=“Description of Artificial Sequence Synthetic peptide”
ITSSGNI

SEQ ID NO: 273 - protein, 7 aa, Artificial Sequence
  source: /note=“Description of Artificial Sequence Synthetic peptide”
ITYPGGT

SEQ ID NO: 274 - protein, 8 aa, Artificial Sequence
  source: /note=“Description of Artificial Sequence Synthetic peptide”
ISSSGEIT

SEQ ID NO: 275 - protein, 10 aa, Artificial Sequence
  source: /note=“Description of Artificial Sequence Synthetic peptide”
MTSAGSGGVA

SEQ ID NO: 276 - protein, 7 aa, Artificial Sequence
  source: /note=“Description of Artificial Sequence Synthetic peptide”
KDNAGVT

SEQ ID NO: 277 - protein, 7 aa, Artificial Sequence
  source: /note=“Description of Artificial Sequence Synthetic peptide”
IDSAGNT

SEQ ID NO: 278 - protein, 7 aa, Artificial Sequence
  source: /note=“Description of Artificial Sequence Synthetic peptide”
ITTGRIT

SEQ ID NO: 279 - protein, 7 aa, Artificial Sequence
  source: /note=“Description of Artificial Sequence Synthetic peptide”
IADDGSA

SEQ ID NO: 280 - protein, 7 aa, Artificial Sequence
  source: /note=“Description of Artificial Sequence Synthetic peptide”
IAAAGNI

SEQ ID NO: 281 - protein, 8 aa, Artificial Sequence
  source: /note=“Description of Artificial Sequence Synthetic peptide”
IYSDGTRT

SEQ ID NO: 282 - protein, 15 aa, Artificial Sequence
  source: /note=“Description of Artificial Sequence Synthetic peptide”
NMKTAWTIGTRGYDY

SEQ ID NO: 283 - protein, 15 aa, Artificial Sequence
  source: /note=“Description of Artificial Sequence Synthetic peptide”
NMKTAWTIGTRGYDY

SEQ ID NO: 284 - protein, 14 aa, Artificial Sequence
  source: /note=“Description of Artificial Sequence Synthetic peptide”
NMRQLPRWSQLLGS

SEQ ID NO: 285 - protein, 14 aa, Artificial Sequence
  source: /note=“Description of Artificial Sequence Synthetic peptide”
NMRQLPRWSLLLGA

SEQ ID NO: 286 - protein, 7 aa, Artificial Sequence
  source: /note=“Description of Artificial Sequence Synthetic peptide”
HLDWTDF

SEQ ID NO: 287 - protein, 14 aa, Artificial Sequence
  source: /note=“Description of Artificial Sequence Synthetic peptide”
NMRLLTTWYNLLGS

SEQ ID NO: 288 - protein, 11 aa, Artificial Sequence
  source: /note=“Description of Artificial Sequence Synthetic peptide”
RIATNVPPYNY

SEQ ID NO: 289 - protein, 14 aa, Artificial Sequence
  source: /note=“Description of Artificial Sequence Synthetic peptide”
NIRILPTWATTVGS

SEQ ID NO: 290 - protein, 16 aa, Artificial Sequence
  source: /note=“Description of Artificial Sequence Synthetic peptide”
TRGGDYSSNDYYGMDY

SEQ ID NO: 291 - protein, 7 aa, Artificial Sequence
  source: /note=“Description of Artificial Sequence Synthetic peptide”
HLDWTDF

SEQ ID NO: 292 - protein, 14 aa, Artificial Sequence
  source: /note=“Description of Artificial Sequence Synthetic peptide”
NIRILPNWATTVGR

SEQ ID NO: 293 - protein, 14 aa, Artificial Sequence
  source: /note=“Description of Artificial Sequence Synthetic peptide”
NMRILPNWNPIVGR

SEQ ID NO: 294 - protein, 14 aa, Artificial Sequence
  source: /note=“Description of Artificial Sequence Synthetic peptide”
NMRLTTSWERLLGA

SEQ ID NO: 295 - protein, 14 aa, Artificial Sequence
  source: /note=“Description of Artificial Sequence Synthetic peptide”
NMRQLPSWSQLLGS

SEQ ID NO: 296 - protein, 17 aa, Artificial Sequence
  source: /note=“Description of Artificial Sequence Synthetic peptide”
NAKKVRSWPITEEPRDY

SEQ ID NO: 297 - protein, 7 aa, Artificial Sequence
  source: /note=“Description of Artificial Sequence Synthetic peptide”
HLDWTDF

SEQ ID NO: 298 - protein, 15 aa, Artificial Sequence
  source: /note=“Description of Artificial Sequence Synthetic peptide”
NMRLSTLSLHRALGA

SEQ ID NO: 299 - protein, 14 aa, Artificial Sequence
  source: /note=“Description of Artificial Sequence Synthetic peptide”
NIRILPRWDQTVGS

SEQ ID NO: 300 - protein, 14 aa, Artificial Sequence
  source: /note=“Description of Artificial Sequence Synthetic peptide”
NMRQLPQWSTLLGA

SEQ ID NO: 301 - protein, 10 aa, Artificial Sequence
  source: /note=“Description of Artificial Sequence Synthetic peptide”
KGSWYFLGDY

SEQ ID NO: 302 - protein, 10 aa, Artificial Sequence
  source: /note=“Description of Artificial Sequence Synthetic peptide”
NFWDGSFNQY

SEQ ID NO: 303 - protein, 11 aa, Artificial Sequence
  source: /note=“Description of Artificial Sequence Synthetic peptide”
NAERVLARQNY

SEQ ID NO: 304 - protein, 17 aa, Artificial Sequence
  source: /note=“Description of Artificial Sequence Synthetic peptide”
NAVVTSRTSLGLRSYVY

SEQ ID NO: 305 - protein, 14 aa, Artificial Sequence
  source: /note=“Description of Artificial Sequence Synthetic peptide”
HTRRWYDDGYDYDT

SEQ ID NO: 306 - protein, 17 aa, Artificial Sequence
  source: /note=“Description of Artificial Sequence Synthetic peptide”
NAVVTSRTSQGLRSYAY

SEQ ID NO: 307 - protein, 6 aa, Artificial Sequence
  source: /note=“Description of Artificial Sequence Synthetic peptide”
NTFDGA

SEQ ID NO: 308 - protein, 13 aa, Artificial Sequence
  source: /note=“Description of Artificial Sequence Synthetic peptide”
NLRRSWAPSDSGF

SEQ ID NO: 309 - protein, 12 aa, Artificial Sequence
  source: /note=“Description of Artificial Sequence Synthetic peptide”
NSDGRFRGIGTY

SEQ ID NO: 310 - protein, 17 aa, Artificial Sequence
  source: /note=“Description of Artificial Sequence Synthetic peptide”
NAVVTSRTSLGLRSYAY

SEQ ID NO: 311 - protein, 13 aa, Artificial Sequence
  source: /note=“Description of Artificial Sequence Synthetic peptide”
NLRRSWAPADNGH

SEQ ID NO: 312 - protein, 12 aa, Artificial Sequence
  source: /note=“Description of Artificial Sequence Synthetic peptide”
KAARSWAYGSGY

SEQ ID NO: 313 - protein, 9 aa, Artificial Sequence
  source: /note=“Description of Artificial Sequence Synthetic peptide”
FAILGGRSY

SEQ ID NO: 314 - protein, 10 aa, Artificial Sequence
  source: /note=“Description of Artificial Sequence Synthetic peptide”
SPAVWTGLRP

SEQ ID NO: 315 - protein, 9 aa, Artificial Sequence
  source: /note=“Description of Artificial Sequence Synthetic peptide”
FAVLDGRSY

SEQ ID NO: 316 - protein, 10 aa, Artificial Sequence
  source: /note=“Description of Artificial Sequence Synthetic peptide”
SPPVWTGLRP

SEQ ID NO: 317 - protein, 8 aa, Artificial Sequence
  source: /note=“Description of Artificial Sequence Synthetic peptide”
FAHYAGYY

SEQ ID NO: 318 - protein, 10 aa, Artificial Sequence
  source: /note=“Description of Artificial Sequence Synthetic peptide”
KVPRFGGSDY

SEQ ID NO: 319 - protein, 10 aa, Artificial Sequence
  source: /note=“Description of Artificial Sequence Synthetic peptide”
KVPRYGGSDY

SEQ ID NO: 320 - protein, 10 aa, Artificial Sequence
  source: /note=“Description of Artificial Sequence Synthetic peptide”
VRSPRTSWTS

SEQ ID NO: 321 - protein, 13 aa, Artificial Sequence
  source: /note=“Description of Artificial Sequence Synthetic peptide”
QAHRWTFDGWQDY

SEQ ID NO: 322 - protein, 10 aa, Artificial Sequence
  source: /note=“Description of Artificial Sequence Synthetic peptide”
VRSPRTSWTS

SEQ ID NO: 323 - protein, 13 aa, Artificial Sequence
  source: /note=“Description of Artificial Sequence Synthetic peptide”
QAHRWTFDGWQDY

SEQ ID NO: 324 - protein, 13 aa, Artificial Sequence
  source: /note=“Description of Artificial Sequence Synthetic peptide”
QARRWTFDGWQDY

SEQ ID NO: 325 - protein, 10 aa, Artificial Sequence
  source: /note=“Description of Artificial Sequence Synthetic peptide”
VRSPRTPWSS

SEQ ID NO: 326 - protein, 10 aa, Artificial Sequence
  source: /note=“Description of Artificial Sequence Synthetic peptide”
ARSPRTSWTV

SEQ ID NO: 327 - protein, 20 aa, Artificial Sequence
  source: /note=“Description of Artificial Sequence Synthetic peptide”
AARWSFGGQCSYGTHNVQRY

SEQ ID NO: 328 - protein, 13 aa, Artificial Sequence
  source: /note=“Description of Artificial Sequence Synthetic peptide”
KARHWEFDGVKEY

SEQ ID NO: 329 - protein, 16 aa, Artificial Sequence
  source: /note=“Description of Artificial Sequence Synthetic peptide”
NRGVLSPSDVYWPSTT

SEQ ID NO: 330 - protein, 7 aa, Artificial Sequence
  source: /note=“Description of Artificial Sequence Synthetic peptide”
AHYGSYY

SEQ ID NO: 331 - protein, 6 aa, Artificial Sequence
  source: /note=“Description of Artificial Sequence Synthetic peptide”
AIGTMA

SEQ ID NO: 332 - protein, 6 aa, Artificial Sequence
  source: /note=“Description of Artificial Sequence Synthetic peptide”
WSALGY

SEQ ID NO: 333 - protein, 13 aa, Artificial Sequence
  source: /note=“Description of Artificial Sequence Synthetic peptide”
QARRWTLDGWQDY

SEQ ID NO: 334 - protein, 13 aa, Artificial Sequence
  source: /note=“Description of Artificial Sequence Synthetic peptide”
QARRWTFDGWQDY

SEQ ID NO: 335 - protein, 10 aa, Artificial Sequence
  source: /note=“Description of Artificial Sequence Synthetic peptide”
ARSPRTSWIL

SEQ ID NO: 336 - protein, 18 aa, Artificial Sequence
  source: /note=“Description of Artificial Sequence Synthetic peptide”
ARRGGAWGSNWWAYHMDD

SEQ ID NO: 337 - protein, 13 aa, Artificial Sequence
  source: /note=“Description of Artificial Sequence Synthetic peptide”
ARYLKLGLSGMDY

SEQ ID NO: 338 - protein, 10 aa, Artificial Sequence
  source: /note=“Description of Artificial Sequence Synthetic peptide”
SRDNFGLGDY

SEQ ID NO: 339 - protein, 6 aa, Artificial Sequence
  source: /note=“Description of Artificial Sequence Synthetic 6xHis tag”
HHHHHH

(19) United States
(12) Patent Application Publication
(10) Pub. No.: US 2022/0242941 A1
(43) Pub. Date: Aug. 4, 2022

(54) ANTIBODIES AGAINST DISEASE CAUSING AGENTS OF POULTRY AND USES THEREOF

(71) Applicant: Novobind Livestock Therapeutics, Inc., Vancouver (CA)

(72) Inventors: Hamlet ABNOUSI, North Vancouver (CA); Slade LOUTET, Shuniah (CA); Filip VAN PETEGEM, Vancouver (CA); Tsz Ying Sylvia CHEUNG, Richmond (CA)

(21) Appl. No.: 17/528,783

(22) Filed: Nov. 17, 2021

Related U.S. Application Data
(63) Continuation of application No. PCT/IB2020/000380, filed on May 19, 2020.
(60) Provisional application No. 62/850,386, filed on May 20, 2019.

(51) Int. Cl. C07K 16/20

(52) U.S. Cl. CPC C07K 16/20; C07K 2317/569; A61K 2039/552; C07K 2317/76; C07K 2317/34; C07K 2317/565

(57) ABSTRACT

Described herein are methods and antibodies useful for reducing, eliminating, or preventing infection with a parasite population in an animal. Also described herein are antigens useful for targeting by heavy chain antibodies and V_{H}H fragments for reducing a parasite population in an animal.

### CROSS-REFERENCE TO RELATED APPLICATIONS

This application is a continuation of International Application No. PCT/IB2020/000380, filed May 19, 2020, which claims the benefit of U.S. Provisional Application No. 62/850,386, filed May 20, 2019, which application is incorporated herein by reference. Priority is claimed pursuant to 35 U.S.C. § 119. The above noted patent application is incorporated by reference as if set forth fully herein.

### SEQUENCE LISTING

The instant application contains a Sequence Listing which has been submitted electronically in ASCII format and is hereby incorporated by reference in its entirety. Said ASCII copy, created Feb. 15, 2022, is named 48647-708_301_SL.txt and is 211,471 bytes in size.

### FIELD OF THE INVENTION

This invention relates to methods and compositions for the control of microorganisms associated with coccidiosis and necrotic enteritis and uses thereof.

### BACKGROUND OF THE INVENTION

Losses to the agriculture industry following contamination of livestock with pathogens are a global burden. With a growing global population and no significant increase in the amount of farm land available to agriculture, there is a need to produce larger quantities of food without using more space. Traditional treatment of animals with antibiotics is a major contributor to the emergence of multi-drug resistant organisms and is widely recognized as an unsustainable solution to controlling contamination of livestock. There is a need for the development of pathogen-specific molecules that inhibit infection or association of the pathogen with the host, without encouraging resistance. Global losses to the poultry industry due to coccidiosis and necrotic enteritis, have been estimated to be €10 billion(1) and $6 billion(2) per annum, respectively.

### SUMMARY OF THE INVENTION

With reference to the definitions set out below, described herein are polypeptides comprising heavy chain variable region fragments (V_{H}Hs) whose intended use includes but is not limited to the following applications in agriculture or an unrelated field: diagnostics, in vitro assays, feed, therapeutics, substrate identification, nutritional supplementation, bioscientific and medical research, and companion diagnostics. Also described herein are polypeptides comprising V_{H}Hs that bind and decrease the virulence of disease-causing agents in agriculture. Further to these descriptions, set out below are the uses of polypeptides that comprise V_{H}Hs in methods of reducing transmission and severity of disease in host animals, including their use as an ingredient in a product. Further described are the means to produce, characterize, refine and modify V_{H}Hs for this purpose.

### BRIEF DESCRIPTION OF THE DRAWINGS

FIG. 1: Illustrates the scientific classification of the Apicomplexa phylum with representative genera and species that cause infections.

FIGS. 2A-2B: Shows a schematic of camelid heavy chain only antibodies and their relationship to V_{H}H domains and complementarity determining regions (CDRs).

FIGS. 3A-3C: Shows phage ELISA binding data for V_{H}H antibodies of this disclosure.

FIG. 4: Shows phage ELISA binding data for V_{H}H antibodies of this disclosure.

### DEFINITIONS

In describing the present invention, the following terminology is used in accordance with the definitions below.

In the following description, certain specific details are set forth in order to provide a thorough understanding of various embodiments. However, one skilled in the art will understand that the embodiments provided may be practiced without these details. Unless the context requires otherwise, throughout the specification and claims which follow, the word “comprise” and variations thereof, such as, “comprises” and “comprising” are to be construed in an open, inclusive sense, that is, as “including, but not limited to.” As used in this specification and the appended claims, the singular forms “a,” “an,” and “the” include plural referents unless the content clearly dictates otherwise. It should also be noted that the term “or” is generally employed in its sense including “and/or” unless the content clearly dictates otherwise. Further, headings provided herein are for convenience only and do not interpret the scope or meaning of the claimed embodiments.

Host

As referred to herein, “host”, “host organism”, “recipient animal”, “host animal” and variations thereof refer to the intended recipient of the product. In certain embodiments, the host is from the superorder Galloanserae. In certain embodiments, the host is a poultry animal. In certain embodiments, the poultry animal is a chicken, turkey, duck, quail, pigeon, squab, pheasant or goose. In certain embodiments, the poultry animal is a chicken. In certain embodiments, the host is a mammal. In certain embodiments, the mammal is a cow, sheep, pig, goat, horse, primate, marsupial, dog, donkey, reindeer, caribou, or deer. In certain embodiments, the mammal is a human. In certain embodiments, the host is an invertebrate.

Pathogens

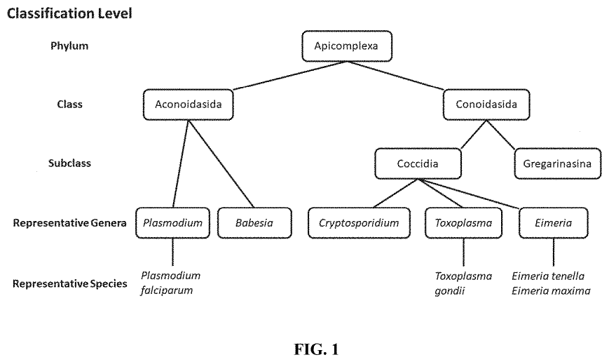

As referred to herein, “pathogen”, “pathogenic”, and variations thereof refer to virulent microorganisms, that can be associated with host organisms, that give rise to a symptom or set of symptoms in that organism that are not present in uninfected host organisms, including the reduction in ability to survive, thrive, reproduce. Without limitation, pathogens encompass parasites, bacteria, viruses, prions, protists, fungi and algae. In certain embodiments, the pathogen is a parasite belonging to the Apicomplexa phylum (FIG. 1). In certain embodiments, the pathogen is a parasite belonging to the Aconoidasida class. In certain embodiments, the pathogen is a parasite belonging to the Plasmodium genus. In certain embodiments, the pathogen is Plasmodium falciparum. In certain embodiments, the pathogen is a parasite belonging to the Babesia genus. In certain embodiments, the pathogen is a parasite belonging to the Conoidasida class. In certain embodiments, the pathogen is a parasite belonging to the Gregarinasina subclass. In certain embodiments, the pathogen is a parasite belonging to the Coccidia subclass. In certain embodiments, the pathogen is a parasite belonging to the Cryptosporidium genus. In certain embodiments, the pathogen is a parasite belonging to the Toxoplasma genus. In certain embodiments, the pathogen is Toxoplasma gondii. In certain embodiments, the pathogen is a parasite belonging to the Eimeria genus. In certain embodiments, the pathogen is Eimeria tenella. In certain embodiments, the pathogen is Eimeria maxima.

“Virulence”, “virulent” and variations thereof refer to a pathogen's ability to cause symptoms in a host organism. “Virulence factor” refers to nucleic acids, plasmids, genomic islands, genes, peptides, proteins, toxins, lipids, macromolecular machineries or complexes thereof that have a demonstrated or putative role in infection.

“Disease-causing agent” refers to a microorganism, pathogen or virulence factor with a demonstrated or putative role in infection.

Parasite

As referred to herein, “parasite”, “parasitic” and variations thereof refer, without limitation, to Eimeria species, or any other parasitic species associated with host organisms. In certain embodiments, bacteria may not be virulent in all host organisms it is associated with.

Antibodies

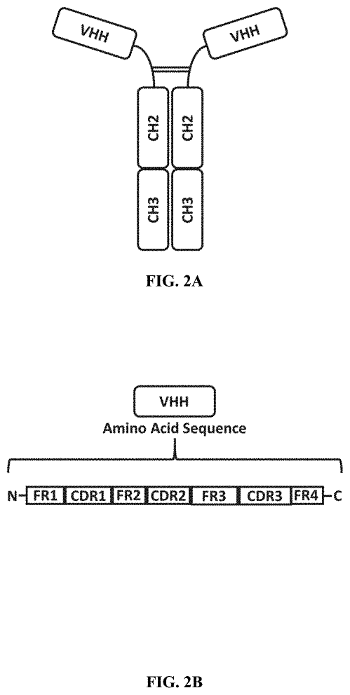

A schematic of camelid heavy chain only antibodies and their relationship to V_{H}H domains and complementarity determining regions (CDRs) is shown in FIG. 2. (Panel A). A camelid heavy chain only antibody consists of two heavy chains linked by a disulphide bridge. Each heavy chain contains two constant immunoglobulin domains (CH2 and CH3) linked through a hinge region to a variable immunoglobulin domain (V_{H}H). (Panel B) are derived from single V_{H}H domains. Each V_{H}H domain contains an amino acid sequence of approximately 110-130 amino acids. The V_{H}H domain consists of the following regions starting at the N-terminus (N): framework region 1 (FR1), complementarity-determining region 1 (CDR1), framework region 2 (FR2), complementarity-determining region 2 (CDR2), framework region 3 (FR3), complementarity-determining region 3 (CDR3), and framework region 4 (FR4). The domain ends at the C-terminus (C). The complementarity-determining regions are highly variable, determine antigen binding by the antibody, and are held together in a scaffold by the framework regions of the V_{H}H domain. The framework regions consist of more conserved amino acid sequences; however, some variability exists in these regions.

As referred to herein “V_{H}H” refers to an antibody or antibody fragment comprising a single heavy chain variable region which may be derived from natural or synthetic sources. NBXs referred to herein are an example of a V_{H}H. In a certain aspect a V_{H}H may lack a portion of a heavy chain constant region (CH2 or CH3), or an entire heavy chain constant region.

As referred to herein “heavy chain antibody” refers to an antibody that comprises two heavy chains and lacks the two light chains normally found in a conventional antibody. The heavy chain antibody may originate from a species of the Camelidae family or Chondrichthyes class. Heavy chain antibodies retain specific binding to an antigen in the absence of any light chain

As referred to herein “specific binding”, “specifically binds” or variations thereof refer to binding that occurs between an antibody and its target molecule that is mediated by at least one complementarity determining region (CDR) of the antibody's variable region. Binding that is between the constant region and another molecule, such as Protein A or G, for example, does not constitute specific binding.

As referred to herein “antibody fragment” refers to any portion of a conventional or heavy chain antibody that retains a capacity to specifically bind a target antigen and may include a single chain antibody, a variable region fragment of a heavy chain antibody, a nanobody, a polypeptide or an immunoglobulin new antigen receptor (IgNAR).

As referred to herein an “antibody originates from a species” when any of the CDR regions of the antibody were raised in an animal of said species. Antibodies that are raised in a certain species and then optimized by an in vitro method (e.g., phage display) are considered to have originated from that species.

As referred to herein “conventional antibody” refers to any full-sized immunoglobulin that comprises two heavy chain molecules and two light chain molecules joined together by a disulfide bond. In certain embodiments, the antibodies, compositions, feeds, products, and methods described herein do not utilize conventional antibodies.

Production System

As referred to herein, “production system” and variations thereof refer to any system that can be used to produce any physical embodiment of the invention or modified forms of the invention. Without limitation, this includes but is not limited to biological production by any of the following: bacteria, yeast, algae, arthropods, arthropod cells, plants, mammalian cells. Without limitation, biological production can give rise to antibodies that can be intracellular, periplasmic, membrane-associated, secreted, or phage-associated. Without limitation, “production system” and variations thereof also include, without limitation, any synthetic production system. This includes, without limitation, de novo protein synthesis, protein synthesis in the presence of cell extracts, protein synthesis in the presence of purified enzymes, and any other alternative protein synthesis system.

Product

As referred to herein, “product” refers to any physical embodiment of the invention or modified forms of the invention, wherein the binding of the V_{H}H to any molecule, including itself, defines its use. Without limitation, this includes a feed, a feed additive, a nutritional supplement, a premix, a medicine, a therapeutic, a drug, a diagnostic tool, a component or entirety of an in vitro assay, a component or the entirety of a diagnostic assay (including companion diagnostic assays).

Feed Product

As referred to herein, “feed product” refers to any physical embodiment of the invention or modified forms of the invention, wherein the binding of the V_{H}H to any molecule, including itself, defines its intended use as a product that is taken up by a host organism. Without limitation, this includes a feed, a pellet, a feed additive, a nutritional supplement, a premix, a medicine, a therapeutic or a drug.

### DETAILED DESCRIPTION OF THE INVENTION

Descriptions of the invention provided are to be interpreted in conjunction with the definitions and caveats provided herein.

For many years, the agriculture industry has utilized antibiotics and anticoccidials to control pathogenic bacteria and parasites, respectively. Some of these molecules also acted as growth promoters. This approach has contributed greatly to the spread of antibiotic and anticoccidial resistance amongst pathogenic organisms. The use of antibiotics as growth promoters in animal feed has already been banned in Europe (effective from 2006) in an effort to phase out antibiotics for non-medicinal purposes and limit antimicrobial resistance. Widespread protection of farmed animals through vaccination has failed due to the short lifespan of many agriculturally important animals, logistical challenges with vaccination of industrial-sized flocks, and high costs. The withdrawal of prophylactic antibiotics and anticoccidials in animal feed and the failure of vaccination to offer widespread protection underpins the need for the development of non-antibiotic products to administer to agricultural animals to prevent infection and promote growth.

Significant pathogens affecting poultry animals include parasites, such as members of the Eimeria genus, as well as bacteria, such as members of the Clostridium and Salmonella genera.

Eimeria parasites, particularly Eimeria tenella, are the causative agent of coccidiosis in chickens. This disease is estimated to cause €10 billion in poultry losses globally(1). Coccidiosis is characterized by reduced weight gain and feed conversion, malabsorption, cell lysis of cells lining the epithelium, and diarrhea(3). Motility, cell adhesion, and tight junction formation are all thought to be important for Eimeria pathogenesis(4).

Intestinal damage caused by Eimeria parasites, particularly Eimeria maxima, is one of the most important predisposing factors for a second disease, chicken necrotic enteritis(5). Losses due to necrotic enteritis are estimated at $6 billion(2) USD per annum. Necrotic enteritis can lead to significant mortality in chicken flocks(3). At subclinical levels, damage to the intestinal mucosa caused by C. perfringens leads to decreased digestion and absorption, reduced weight gain and increased feed conversion ratio (6).

Prior arts relating to the field of this invention rely on the host organism to generate protection against disease-causing agents. This approach is often limited by the short lifespan of the host organisms affected by the pathogens listed above, which do allow the host organism's immune system sufficient time to generate long-lasting immunity. Furthermore, the effectiveness of prior arts is limited by technical challenges associated with widespread vaccination of large flocks of host organisms. These problems are circumvented by introducing exogenous peptides that neutralise the virulence and spread of the disease-causing agent into the host via feed without eliciting the host immune response. Moreover, the methods described herein provide scope for the adaptation and refinement of neutralising peptides, which provides synthetic functionality beyond what the host is naturally able to produce.

Antibody heavy chain variable region fragments (V_{H}Hs) are small (12-15 kDa) proteins that comprise specific binding regions to antigens. When introduced into an animal, V_{H}Hs bind and neutralise the effect of disease-causing agents in situ. Owing to their smaller mass, they are less susceptible than conventional antibodies, such as previously documented IgYs, to cleavage by enzymes found in host organisms, more resilient to temperature and pH changes, more soluble, have low systemic absorption and are easier to recombinantly produce on a large scale, making them more suitable for use in animal therapeutics than conventional antibodies.

Antibodies for Preventing or Reducing Virulence (Summary)

In one aspect, the present invention provides a polypeptide or pluralities thereof comprising a V_{H}H or V_{H}Hs that bind disease-causing agents to reduce the severity and transmission of disease between and across species. In certain embodiments, the V_{H}H is supplied to host animals. In certain embodiments, the V_{H}H is an ingredient of a product.

Binding to Reduce Virulence

In another aspect, the present invention provides a polypeptide or pluralities thereof comprising a V_{H}H or V_{H}Hs that bind disease-causing agents, and in doing so, reduce the ability of the disease-causing agent to exert a pathological function or contribute to a disease phenotype. In certain embodiments, binding of the V_{H}H(s) to the disease-causing agent reduces the rate of replication of the disease-causing agent. In certain embodiments, binding of the V_{H}H(s) to the disease-causing agent reduces the ability of the disease-causing agent to bind to its cognate receptor. In certain embodiments, binding of the V_{H}H(s) to the disease-causing agent reduces the ability of the disease-causing agent to interact with another molecule or molecules. In certain embodiments, binding of the V_{H}H(s) to the disease-causing agent reduces the mobility or motility of the disease-causing agent. In certain embodiments, binding of the V_{H}H(s) to the disease-causing agent reduces the ability of the disease-causing agent to reach the site of infection. In certain embodiments, binding of the V_{H}H(s) to the disease-causing agent reduces the ability of the disease-causing agent to cause cell death.

Antibodies Derived from Llamas

In a further aspect, the present invention provides a method for the inoculation of Camelid or other species with recombinant virulence factors, the retrieval of mRNA encoding V_{H}H domains from lymphocytes of the inoculated organism, the reverse transcription of mRNA encoding V_{H}H domains to produce cDNA, the cloning of cDNA into a suitable vector and the recombinant expression of the V_{H}H from the vector. In certain embodiments, the camelid can be a dromedary, camel, llama, alpaca, vicuna or guacano, without limitation. In certain embodiments, the inoculated species can be, without limitation, any organism that can produce single domain antibodies, including cartilaginous fish, such as a member of the Chondrichthyes class of organisms, which includes for example sharks, rays, skates and sawfish. In certain embodiments, the heavy chain antibody comprises a sequence set forth in Table 1. In certain embodiments, the heavy chain antibody comprises an amino acid sequence with at least 80%, 90%, 95%, 97%, or 99% identity to any sequence disclosed in Table 1. In certain embodiments, the heavy chain antibody possess a CDR1 set forth in Table 2. In certain embodiments, the heavy chain antibody possess a CDR2 set forth in Table 2. In certain embodiments, the heavy chain antibody possess a CDR3 set forth in Table 2.

TABLE 1
Unique SEQ IDs for VHH antibodies of this disclosure

| SEQ ID | NBX | Amino acid sequence | Antigen |
|---|---|---|---|
| 1 | NBX0707 | QVQLQESGGGLVQAGGSLRLSCAASGSIFSRDTMA | EmMIC2 |
| | | WYRQVPGEQRELVAFITNFGGTNHADSVKGRFTIS | |
| | | RDNAKNTVYLQMNSLKPEDTAVYYCAAGLVSLRT | |
| | | HTWEYWGQGTQVTVSS | |
| 2 | NBX0708 | QVQLQESGGGLVQAGDSLSLSCVDSKRTADRYVM | EmMIC2 |
| | | HWFRQVPGKDREFVAAISGNGLVRNYADSVKGRF | |
| | | TISRDNAKNMVYLQMKSLKPEDTAVYYCAADFVL | |
| | | DSRWRYVPYWGQGTQVTVSS | |
| 3 | NBX0709 | QVQLQESGGGLVQAGGSLRLSCAASGSIFSRDTMA | EmMIC2 |
| | | WYRQVPGEQRELVAYITNFGGTNHADSVKGRFTIS | |
| | | RDNAKNTVYLQMNSLKPEDTAVYYCAAGLVSLHT | |
| | | HTWEYWGQGTQVTVSS | |
| 4 | NBX0710 | QVQLQESGGGLVQAGGSLRLSCAASGSIFSINAMG | EmMIC2 |
| | | WYRQAPGKQRELVAGITSGGSTNYADSVKGRFTIS | |
| | | RDNAKNTVYLQMNSLKPEDTAVYYCAADYVGLG | |
| | | DDAFYVPYWGQGTQVTVSA | |
| 5 | NBX0711 | QVQLQQSGGGLVQPGGSLRLSCAASRSIRSADAMA | EmMIC2 |
| | | WYRQAPGKQRVWVATITSGGSTYYTDSVKGRFTIS | |
| | | RDNAKSTVYLQMSSLKPEDTAVYYCNAVGDRTSY | |
| | | WGQGTQVTVAS | |
| 6 | NBX0712 | QVQLQESGGGLVQTGGSLRLSCAASGRTFTRNAM | EmMIC2 |
| | | GWFRQAPGKAREFVAVISWSGNRAYSDSVKGRFTI | |
| | | SRDNGKNLVSLQMNSLKPEDTAVYYCAAAREAITS | |
| | | TYYTPHVLTDYDAWGQGAQVTVSS | |
| 7 | NBX0713 | QVQLQESGGGLVQPGGSLRLSCAASGRTFRRNAM | EmMIC2 |
| | | GWFRQAPGKAREFVATISWSGNRAYSDSVKGRFTI | |
| | | SRDNGKNLVSLQMNSLKPEDTAVYYCAAARERITS | |
| | | TYYTPHVLTDYDAWGQGAQVTVSS | |
| 8 | NBX0714 | QVQLQESGGGLVQAGGSLRLSCAASGSIFSRDTMA | EmMIC2 |
| | | WYRQVPGEQRELVAYITNFGGTNHAGSVKGRFTIS | |
| | | RDNAKNTVYLQMNSLKPEDTAVYYCAAELVSLRT | |
| | | HTWEYWGQGTQVTVSS | |
| 9 | NBX0715 | QVQLQQSGGGLVQAGGSLRLSCAASGRTFSRNAM | EmMIC2 |
| | | GWFRQAPGKAREFVATISWSGNRAYSDSVKGRFTI | |
| | | SRDNEKNLVSLHMNSLKPEDTAVYYCAAARERITS | |
| | | TYYTPHVLTDYDAWGQGAQVTVSS | |
| 10 | NBX0716 | QVQLQESGGGLVQAGGSLSLSCTASGRTFSTFPVA | EmMIC2 |
| | | WFRQAPGKEREFVARINWFTTDTYYADSVKGRFTI | |
| | | SRDSAKNTVYLQMNALKPEDTAIYYCAAARSNTG | |
| | | TSRFDYWGQGTQVTVSS | |
| 11 | NBX0717 | QVQLQESGGGLVQAGGSLRLSCAASTNIATFTTMG | EmMIC2 |
| | | WYRQAPGKERELVATTTPWGATTSYADSVKGRFTI | |
| | | SNDNAKNTVNLQMNSLKPEDTAVYYCNAQQDIPT | |
| | | TQTYWGQGTQVTVSS | |
| 12 | NBX0718 | QVQLQESGGGLVQAGGSLRLSCAANGRTFSSYAM | EmMIC2 |
| | | AWFRQAPGKEREFVAAVGWSGGRTYYTDSVKGRF | |
| | | TISRDNAKDTVYLQMNSLKPEDTAVYYCAATRGW | |
| | | AQATLLYDYDYWGQGTQVTVSS | |
| 13 | NBX0726 | QVQLQQSGGGLVQAGGSVRLSCTANGLTFGNYAM | EtMIC2 |
| | | AWFRRTPGKERAFVGGMSASGAGTYYLDSVKGRF | |
| | | TISRDTAKNTVYLEMNSLKAEDTAVYYCAANSIYP | |
| | | GRRWASYDYWGQGTQVTVSS | |
| 14 | NBX0727 | QVQLQQSGGGLVQAGGSVRLSCAASGLTLGNYAL | EtMIC2 |
| | | AWFRRTPGKEREFVAGMSGSGAGTYYLDSVNGRF | |
| | | TISRDNAKNTLYLQMNSLKAEDTAVYYCAANSIYP | |
| | | GRRWASYDYWGQGTQVTVSS | |
| 15 | NBX0728 | QVQLQESGGGLVQAGGSLRLSCAASASIFSRDTMA | EtMIC2 |
| | | WYRQVPGEQRELVAYVTNFGGTNHADSVKGRFTI | |
| | | SRDNAKNTVYLQMNSLKPEDTAVYYCAADLISLRT | |
| | | HTWEYWGQGTQVTVSS | |
| 16 | NBX0731 | QVQLQESGGGLVQPGGSLRVSCAASGFTFSRDTMA | EmMIC2 |
| | | WYRQVPGEQRELVAYITNFGGTNHADSVKGRFTIS | |
| | | RDNAKNTVYLQMNSLKPEDTAVYYCAAGLVSLRT | |
| | | HTWEYWGQGTQVTVSS | |
| 17 | NBX0732 | QVQLQESGGGLVQTGGSLRLSCAASGRTFRRNAM | EmMIC2 |
| | | GWFRQAPGKAREFVATITWSGNRAYSDSVKGRFTI | |
| | | SRDNEKNLVSLQMNSLKPEDTAVYYCAAARERITS | |
| | | TYYTPHVLTEYDAWGQGAQVTVSS | |
| 18 | NBX0733 | QVQLQQSGGGLVQPGGSLRLSCAASGSIFSVDAMA | EmMIC2 |
| | | WYRQAPGKQREWVATFTKGGSTYYAGSVKGRFTI | |
| | | SRDNAKNTVYLQMSSLKPEDTAVYYCNAVGDRTG | |
| | | AWGQGTQVTVAS | |
| 19 | NBX0734 | QVQLQESGGGLVQAGGSLRLSCAASGSIFSRDTMA | EmMIC2 |
| | | WYRQVPGEQRELVAYITNFGGTNHADSVKGRFTIS | |
| | | RDNAKNTVYLQMNSLKPEETAVYYCAAGLVSLRT | |
| | | HTWEYWGQGTQVTVSS | |
| 20 | NBX0740 | QVQLQESGGGLVQAGGSLRLSCAASGRTFVNYNM | EmMIC2 |
| | | GWFRQAPGKEREFVATINVVSGGSTYYAGSVKGRF | |
| | | TISRDSAKNTVYLQMNKLKPEDTAVYYCAAEVGY | |
| | | GYQGPPLTTPSMYDYWGQGTQVTVST | |
| 21 | NBX0748 | QVQLQESGGGLVQAGGSLRLSCVASGSIFSRDTMA | EtMIC2 |
| | | WYRQVPGEQRELVAYITNFGGTNHADSVKGRFTIS | |
| | | RDNAKNTVYLQMNSLKPEDTAVYYCAAGLLSLRS | |
| | | HTWELWGQGTQVTVSS | |
| 22 | NBX0749 | QVQLQESGGGLVQAGGSLRLSCATSGSIFSRDTMA | EtMIC2 |
| | | WYRQVPGEQRELVAYITNFGGTNHADSVKGRFTIS | |
| | | RDNAKNTVYLQMNSLKPEDTAVYYCAAGLLSLRT | |
| | | HTWEYWGQGTQVTVSS | |
| 23 | NBX0750 | QVQLQESGGGLVQPGDSLRLSCAPSGRTFSGYVTG | EmMIC2 |
| | | WFRQAPGMEREFVAAITWSGDSTYYADSVKGRFQI | |
| | | SRDSAKNTVYLQMNSLKPEDAGVYYCAVKSQTYS | |
| | | TDYVQPRRYAYWGQGTQVTVSS | |
| 24 | NBX0751 | QVQLQESGGGLVQPGGSLRLSCAVSGTIFSITPMG | EmMIC2 |
| | | WYRQAPGKQRELVASISGGGSTNYTDPVKGRFTIS | |
| | | RDKARNTVYLQMNNLKPEDTAVYYCNAASLAIVR | |
| | | GINNYWGQGTQVTVSS | |
| 25 | NBX0752 | QVQLQESGGGLVQAGGSLRLSCAASGSIFSRDNMA | EmMIC2 |
| | | WYRQVPGEQRELVALITNFGGTNHADSVKGRFTIS | |
| | | RDNAKNTVYLQMNSLKPEDTAVYYCAAGLVSLLT | |
| | | HTWEYWGQGTQVTVSS | |
| 111 | NBX16011 | QVQLQESGGGLVQAGGSLRLSCKISGNTFSSYTMG | EmAMA1 |
| | | WYRRPPGKQRELVAQLTKGGSTNYADSVKDRFTIS | |
| | | TDNAKNTVYLQMDSLKPEDTAVYYCNMKTAWTI | |
| | | GTRGYDYWGQGSQVTVSS | |
| 112 | NBX16012 | QVQLQESGGGLVQAGGSLRLSCAISRSTFSSYTMG | EmAMA1 |
| | | WYRRPPGKERELVAQITNGGSTNYADSVKGRFTIS | |
| | | RDNAKNTVYLQMNSLKPEDTAVYYCNMKTAWTI | |
| | | GTRGYDYWGQGSQVTVSS | |
| 113 | NBX16013 | QVQLQESGGGLVQAGGSLRLSCVASGSSISSYAMA | EmAMA1 |
| | | WYRQAPGQQRELVAGISSGGATEYADSAKARFTIS | |
| | | RDNAKNTVYLQLYNLKPEDTAVYYCNMRQLPRW | |
| | | SQLLGSWGQGTQVSVSS | |
| 114 | NBX16014 | QVQLQQSGGGLVQAGGSLRLSCSASGSTISSYAMA | EmAMA1 |
| | | WYRQAPGQQRELVAGISSGGGTQYADSAKGRFTIS | |
| | | RDNAKNTVYLQLHTLKPEDTAVYYCNMRQLPRWS | |
| | | LLLGAWGQGTQVSVSS | |
| 115 | NBX16015 | QVQLQQSGGGLVQAGGSLRLSCAASGSTLTRYSVS | EmAMA1 |
| | | WYRQAPGNEREVVSRILKGGSTHYADSVKGRFTIS | |
| | | RDNAKNTVSLQMNSLKPEDTAVYYCHLDWTDFW | |
| | | GQGTQVTVSS | |
| 116 | NBX16016 | QVQLQQSGGGLVQAGGSLRLSCAASGSSFSSYAM | EmAMA1 |
| | | GWYRQAPGKQREWVAGIGSLGSPNYADSVKGRFV | |
| | | MSRDNAKNTVYLQMNSLKPEDTAVYYCNMRLLT | |
| | | TWYNLLGSWGQGTQVTVSS | |
| 117 | NBX16017 | QVQLQESGGGLVEAGGSLRLSCVASRNIFGVAHMT | EmAMA1 |
| | | WYRQAPGKERELVATVSSSGTTNHVDSVKGRFTIS | |
| | | RDNSKTALYLQMNTLKPEDTAVYVCRIATNVPPYN | |
| | | YVVGQGIQVTVSS | |
| 118 | NBX16018 | QVQLQESGGGLVQPGGSLRLSCAASGSSISNYAMA | EmAMA1 |
| | | WYRQAPGKQREWVAGISQRMDTIYADSVKGRFTIS | |
| | | RDNAKNTVSLQMNSLKPEDTAVYYCNIRILPTWAT | |
| | | TVGSWGQGTQVTVSS | |
| 119 | NBX16019 | QVQLQESGPGLVKPSQTLSLTCTVSGGSITTNRYY | EmMIC1 |
| | | WSWIRQPPGKGLEWMGAIAYDGSTYYSPSLKSRTA | |
| | | ISRDTSKNQFSLQLSSVTPEDTAVYYCTRGGDYSSN | |
| | | DYYGMDYWGKGTLVTVSS | |
| 120 | NBX16020 | QVQLQESGGGLVQAGGSLRLSCAASGSSLSRYSVS | EmAMA1 |
| | | WYRQAPGDEREVVSRLTSRGDNFYADSVKGRFTIS | |
| | | RDNAKNTVYLQMNSLKPEDTAVYYCHLDWTDFW | |
| | | GQGTQVTVSS | |
| 121 | NBX16021 | QVQLQESGGGLVQAGGSLRLSCVASGSDVSTYAL | EmAMA1 |
| | | GWYRQAPGKQREWVAGISRGGDTNYADSVKGRF | |
| | | TISRDYAKNTVYLQMNSLKPEDTAVYYCNIRILPN | |
| | | WATTVGRWGQGTQVTVSS | |
| 122 | NBX16022 | QVQLQESGGGLVQAGGSLRLSCVASGSSGSDYALG | EmAMA1 |
| | | WYRQAPGKQREWVAGISRGGDTNYADSVQGRFTI | |
| | | SRDNAKNTVYLQMNSLKPEDTAVYYCNMRILPNW | |
| | | NPIVGRWGQGTQVTVSS | |
| 123 | NBX16023 | QVQLQESGGGLVQAGGSLRLSCAAPVRTFSNYAM | EmAMA1 |
| | | GWYRQAPGKQRELVAGISSNGRTDYVDSVKGRFTI | |
| | | SRDNAKNTVSLQMNSLKPEDTAVYYCNMRLTTSW | |
| | | ERLLGAWGQGTQVTVSS | |
| 124 | NBX16024 | QVQLQESGGGLVQAGGSLRLSCSASGSTFSTFSRY | EmAMA1 |
| | | AMAWYRQAPGQQREWVAGISSGGSTTYADSAKG | |
| | | RFTISRDNAKNTVYLQLHSLKPEDTAVYYCNMRQL | |
| | | PSWSQLLGSWGQGTQVSVSS | |
| 125 | NBX16025 | QVQLQESGGGLVQTGGSLRLACAASGDSFILYTAG | EmAMA1 |
| | | WYRQAPGKERELVAQITSGGTTNYGDFAKGRFTIF | |
| | | RDDAKNMVILQMASLKPEDTAVYYCNAKKVRSW | |
| | | PITEEPRDYWGQGTQVTVSS | |
| 126 | NBX16026 | QVQLQESGGGLVQPGGSLRLSCAASGSTLTRYSVS | EmAMA1 |
| | | WYRQAPGNERDVVARILKGGSTHYSDSVKGRFTIS | |
| | | RDNAKNTVSLQMNSLGPEDTAVYYCHLDWTDFW | |
| | | GPGTQVTVSS | |
| 127 | NBX16027 | QVQLQESGGGLVQAGGSLRLSCAASGSSISSYAMG | EmAMA1 |
| | | WYRQAPGKQREWVAVIGSGGTTWYADSVKGRSTI | |
| | | SRDNAKNTVYLQMNSLKPEDTAVYYCNMRLSTLS | |
| | | LHRALGAWGQGTRVTVSS | |
| 128 | NBX16028 | QVQLQESGGGLVQAGGSLRLSCSASGSTFTSSGSTF | EmAMA1 |
| | | SRYAMAWYRQATGQQRELVAGISSGGSTQYADFA | |
| | | RGRFTISRDNAKDTVYLQMNNLQPEDTAVYYCNIR | |
| | | ILPRWDQTVGSWGQGTQVTVSS | |
| 129 | NBX16029 | QVQLQESGGGLVQAGGSLRLSCVASGSIFTAGSTF | EmAMA1 |
| | | NTYAMAWYRQAPGQSRELVAGISSSGSTEYADSV | |
| | | KGRFTISRDNAKNTVYLQLHSLKPEDTAVYYCNM | |
| | | RQLPQWSTLLGAWGQGTQVSVSS | |
| 130 | NBX16030 | QVQLQQSGGGLVQAGGSLTLSCAISGNTYNAHTM | EmMIC1 |
| | | AWYRQAPGKQRELVARMDFNGESNYDNSVKGRF | |
| | | TISRDNAENTLFLQMNSLRPDDTAVYYCKGSWYFL | |
| | | GDYWGQGTQVTVSS | |
| 131 | NBX16033 | QVQLQESGGGTVQAGGSLRLSCAVSGSTFSSYSMN | EtRON2 |
| | | WYRQGPGNQREWVGGIRSNDSTYYADSVKGRFTI | |
| | | SRDNAKNAWNLQMNSLKSEDTAVYYCNFWDGSF | |
| | | NQYWGQGTQVTVSA | |
| 132 | NBX16034 | QVQLQESGGGVVQAGGSLRLSCVASGSDTSVYAM | EtRON2 |
| | | RWWRQVPGKERLLLASITSGGTVRYAESVKGRFTI | |
| | | SRDDAKNTLYLQMNSLKTEDTAVYLCNAERVLAR | |
| | | QNYWGQGTQVTVSS | |
| 133 | NBX16035 | QVQLQESGGGLVQAGGSLRLSCAASGSIFSINVIGW | EtRON2 |
| | | YRQVPGKERELVANSGSTTKYADSVKGRFTISRDN | |
| | | AKNTVDLQMNSLKPEDTAVYYCNAVVTSRTSLGL | |
| | | RSYVYWGQGTQVTVSS | |
| 134 | NBX16036 | QVQLQESGGGLVQAGGSLRLSCVALGNIGWINDM | EtRON2 |
| | | GWYRQAPGKERELVARISSAEATNYADAVKGRFTI | |
| | | SRDDAKNTVYLQMNSLKPEDTAVYSCHTRRWYD | |
| | | DGYDYDTWGQGTQVTVSS | |
| 135 | NBX16037 | QVQLQQSGGGLVQAGGSLRLSCAASGSIFSINVIGW | EtRON2 |
| | | YRQVPGKERELVAKSDRTTKYADSVKGRFTISRDN | |
| | | AKNTVDLQMNSLKPEDTAVYYCNAVVTSRTSQGL | |
| | | RSYAYWGQGTQVTVSS | |
| 136 | NBX16038 | QVQLQQSGGGLVQSGGSLRLSCTATRSTISGYGGR | EtRON2 |
| | | WYRQAPGKQREFVALLSSGGMTRYADSVMGRFTI | |
| | | SRDDIKNTLYLQMNTLKPEDTAVYYCNTFDGAWG | |
| | | QGTQVTVSS | |
| 137 | NBX16039 | QVQLQQSGGGLVQAGGSLRLSCTTSGIIFSFSLLNV | EtRON2 |
| | | GWYRQAPGKERELVARMSTAGNTYYSTSVKDRFT | |
| | | ISRDNGKNTVDLQMNSLNADDTAVYYCNLRRSWA | |
| | | PSDSGFWGQGTQVFVSS | |
| 138 | NBX16040 | QVQLQESGGGMVQAGGSLRLSCAASGTTITINTMR | EtRON2 |
| | | WYRQPPGKERELVASIVPSGKTYYADSVKGRFTISR | |
| | | DNHKNTMYLQMNSLKPEDTAVYYCNSDGRFRGIG | |
| | | TYWGQGTQVTVSS | |
| 139 | NBX16041 | QVQLQESGGGLVQAGGSLRLSCAASGSIFSINVIGW | EtRON2 |
| | | YRQAPGKERELVANSGSTTKYADSVKGRFTISRDN | |
| | | AKNTVDLQMNSLKPEDTAVYYCNAVVTSRTSLGL | |
| | | RSYAYWGQGTQVTVSS | |
| 140 | NBX16042 | QVQLQESGGGLVQAGGSLRLSCVASGITFNFNYYV | EtRON2 |
| | | MGWYRQAPGKQRELVARISDGGNTNYADSVKGRF | |
| | | TISRDNTKNTGYLQMNSLKPEDTAIYYCNLRRSWA | |
| | | PADNGHWGQGTQVTVSS | |
| 141 | NBX16043 | QVQLQESGGGLVQAGGSLRLSCAASGIAFSMFDM | EtRON2 |
| | | VWYRQAPGKQRELVASVSDGGSTAYADSVKGRFT | |
| | | ISRDNAKKMVYLQMNSLKPEDTAVYYCKAARSW | |
| | | AYGSGYWGQGIQVTVSS | |
| 142 | NBX17001 | QVQLQQSGGGLVQAGGSLRLSCAASGITPSRHPMT | EtAMA1 |
| | | WYRQVPGKKRELVASLTDADSTYYTDSVKGRFTIL | |
| | | RDSAANTVYLQLNGLKPEDTAVYYCFAILGGRSY | |
| | | WGQGTQVTVSS | |
| 143 | NBX17002 | QVQLQESGGGLVQAGGSLRLSCAASQSITSTYGMA | EtAMA1 |
| | | WYRQAPGKQREWVATINHTGNTNYVASVKGRFAI | |
| | | SRDNSDYTVYLQMSDLKPEDTAVYYCSPAVWTGL | |
| | | RPWGQGTQVTVSS | |
| 144 | NBX17003 | QVQLQESGGGLVQAGGSLRLSCAASGSTPSRHPMT | EtAMA1 |
| | | WYRQAPGKSRELLASITNVDSTYYADSVKGRATIF | |
| | | RDNAASTVYLQLNGLKLEDTAVYSCFAVLDGRSY | |
| | | WGQGTQVTVSS | |
| 145 | NBX17004 | QVQLQESGGGLVNPGGSLRLSCAASVSTTSTYGMT | EtAMA1 |
| | | WYRQAPGKQREWVATINHTGGTNYADSVKGRVAI | |
| | | SIDNAKNTVYLQMSDLKPEDTAVYYCSPPVWTGL | |
| | | RPWGQGTQVTVSS | |
| 146 | NBX17005 | QVQLQESGGGTVQAGESLTLSCAASGTIFRFTVMG | EtAMA1 |
| | | WYRQVPGKEREFVASITYTDATDYADSVKGRFTIS | |
| | | RDNAKNTAYLQMNSLKPEDTAVYSCFAHYAGYY | |
| | | YGQGTQVTVSP | |
| 147 | NBX17006 | QVQLQQSGGGLVQAGGSLRLSCTASGITSSTYAMS | EtAMA1 |
| | | WYRQAPGKEREPVASMASSGSTFYADSVKGRFTIS | |
| | | RDNAKNMVYLQMNSLKPGDTAVYYCKVPRFGGS | |
| | | DYVVGQGTQVTVSS | |
| 148 | NBX17007 | QVQLQQSGGGLVQPGGSLRLSCTASGITSSTYAMS | EtAMA1 |
| | | WYRQAPGKQREPVASIASSGSTFYAESVKGRFTISR | |
| | | DNAKNMAFLQMNSLEPGDTAVYYCKVPRYGGSD | |
| | | YVVGQGTQVTVSS | |
| 149 | NBX17008 | QVQLQESGGGLVQAGGSLRLSCAASRSSLSTYAMG | EtMIC1 |
| | | WYRQAPGKQRELIATITTGGVTQYVDFVKGRFTIS | |
| | | RDNAKNTVYLQMNSLKPEDTAVYYCVRSPRTSWT | |
| | | SWGQGTQVTVSS | |
| 150 | NBX17009 | QVQLQQSGGGLVQAGGSLRLSCVASASALRMGW | EtMIC1 |
| | | YRQAPGEQRELVATLDNAGKTNYAASVKGRFTISR | |
| | | DSAKNTVYLQMNSLKPEDTAVYYCQAHRWTFDG | |
| | | WQDYWGQGTQVTVSS | |
| 151 | NBX17010 | QVQLQESGGGLVQAGGSLRLSCTASRSTLSTYAMG | EtMIC1 |
| | | WYRQAPGEQRELVATITTGHITQYADFVKGRFTISR | |
| | | DNTKNTVYLQMNSLKPEDTAVYYCVRSPRTSWTS | |
| | | WGQGTQVTVSS | |
| 152 | NBX17011 | QVQLQESGGGLVQAGGSLRLSCAASASALRMGWY | EtMIC1 |
| | | RQAPGESRELVATIDNAGKTNYADSVKGRFTIAKD | |
| | | SAKNTVYLQMNSLKPGDTAVYYCQAHRWTFDGW | |
| | | QDYWGQGTQVTVSS | |
| 153 | NBX17012 | QVQLQESGGGLVQAGGSLRLSCASSASALRMGWY | EtMIC1 |
| | | RQAPGEQRELVATMDNAGSTNYAGSVKGRFTISRD | |
| | | SAKNTVYLQMNSLKPGDTAVYYCQARRWTFDGW | |
| | | QDYWGQGTQVTVSS | |
| 154 | NBX17013 | QVQLQQSGGGLVQAGGSLRLSCAASGSTLSTYAM | EtMIC1 |
| | | GWYRQAPGKQRELVATITTGRVTQYADFVKGRFTI | |
| | | SRDNAKNTVYLQMNSLKPEDTAVYYCVRSPRTPW | |
| | | SSWGQGTQVTVSS | |
| 155 | NBX17014 | QVQLQQSGGGLVQAGGSLRLSCATRSSLSTYAMG | EtMIC1 |
| | | WYRQAPGKQRELVATITTGHITQYADFVKGRFTIS | |
| | | RDNAKNMVYLQMNSLKPEDTAVYYCARSPRTSW | |
| | | TVWGQGTQVTVSS | |
| 156 | NBX17015 | QVQLQESGGGLVQAGGSLRLSCAASGFSLASYHIT | EtMIC1 |
| | | WFRQAPGKEREGVSCIGYNSGWTDYGEFVKGRFTI | |
| | | SRDNDKNTVYLQMNNLQPEDSAVYYCAARWSFG | |
| | | GQCSYGTHNVQRYRGQGTQVTVSS | |
| 157 | NBX17016 | QVQLQQSGGGKVQPGGSLRLSCVASRPVFSMAWY | EtMIC1 |
| | | RQAPGKQRVMVASTTNGKEPNYEDSVQGRFTISRD | |
| | | NAKNAVYLQMNSLKPEDTAIYSCKARHWEFDGVK | |
| | | EYWGQGTQVTVSS | |
| 158 | NBX17017 | QVQLQESGGGLGQAGGSLRLSCEASGRAFSTYHM | EtAMA1 |
| | | GWYRQAPGKQRELVATITSSGNINYADSVKGRFTIS | |
| | | RDNAKNTVNLQMNNLKPDDTAVYYCNRGVLSPSD | |
| | | VYWPSTTWGQGTQVTVSS | |
| 159 | NBX17018 | QVQLQESGGGTVQAGESLTLSCAASGSIFRFTVMG | EtAMA1 |
| | | WYRQVPGKEREFVASITYPGGTEYVDSVKGRFTIS | |
| | | RDNAKNTAYLQVSSLKPEDTAVYYCFAHYGSYYY | |
| | | GQGTQVTVSP | |
| 160 | NBX17019 | QVQLQESGGGLVQAGGSLRLSCTGPGSTFSSYAVG | EmRON2 |
| | | WYRQAPGGNREWVASISSSGEITRYADSVTGRFTIS | |
| | | RDNAKNTVDLQMNSLRPEDTAVYYCAIGTMARGK | |
| | | GTLVTVSS | |
| 161 | NBX17020 | QVQLQQSGGGLVQAGGSLRLSCAGSGRPFGSYVM | EmRON2 |
| | | GWYRQAPGEQREMVARMTSAGSGGVADYGESVK | |
| | | GRFAISRDYAKNMVFLQMNSLKPEDTAVYYCWSA | |
| | | LGYWGQGTQVTVSS | |
| 162 | NBX17021 | QVQLQQSGGGLVQAGGSLRLSCAASRSALSMGWY | EtMIC1 |
| | | RQAPGEQRELVATKDNAGVTTYADSVKGRFTVSR | |
| | | DSAKNTVYLQMNSLKPEDTAVYYCQARRWTLDG | |
| | | WQDYWGQGTQVTVSS | |
| 163 | NBX17022 | QVQLQESGGGLVQAGGSLRLSCAASASALRMGWY | EtMIC1 |
| | | RQAPGEQRELVATIDSAGNTNYAGSVKGRFTISRDS | |
| | | AKNTVYLQMNSLKPGDTAVYYCQARRWTFDGWQ | |
| | | DYVVGQGTQVTVSS | |
| 164 | NBX17023 | QVQLQESGGGLVQAGGSLRLSCTASRSTLSTYAMG | EtMIC1 |
| | | WYRQAPGKQRELVATITTGRITQYADFVKGRFTISR | |
| | | DNDKNTVYLQMNSLKPEDTAVYYCARSPRTSWIL | |
| | | WGQGTQVTVSS | |
| 165 | NBX17024 | QVQLQESGPGLVKPSQTLSLTCTVSGGSITTSYSTW | EmRON2 |
| | | SWIRQPPGKGLEWMGVIADDGSAEYSPSLKSRTSIS | |
| | | LDTSKNQFSLQLSSVTPEDTAVYYCARRGGAWGS | |
| | | NVVWAYHMDDWGKGTLVTVSS | |
| 166 | NBX17034 | QVQLQESGPSLVRPSQTLTLTCTLSGGSITDDHYY | EtMIC1 |
| | | FTWIRQLPGKELEWLGTIAAAGNIFPSPSFESRTS | |
| | | ISRDTSSNQFTLRLNSATPEDTAVYYCARYLKLG | |
| | | LSGMDYVVGKGILVTVSS | |
| 167 | NBX17035 | QVQLQESGGGLVQPGGSLRVSCVASGFTFSAAYMS | EtMIC1 |
| | | WVRQAPGKGLEWVSTIYSDGTRTYYADSVKGRFTI | |
| | | SRDNTKNTVYLEMNSLKPEDTALYYCSRDNFGLG | |
| | | DYVVGQGTQVTVSS | |

TABLE 2
Unique SEQ IDs for V_{H}H CDRs of this disclosure

| | | | | CDR2 | | CDR3 | |
|---|---|---|---|---|---|---|---|
| | CDR1 | CDR1 | CDR2 | SEQ | CDR3 | SEQ | |
| | Amino Acid | SEQ ID | Amino Acid | ID | Amino Acid | ID | |
| NBX | Sequence | NO: | Sequence | NO: | Sequence | NO: | Antigen |
| NBX0707 | GSIFSRDT | 26 | ITNFGGT | 51 | AADFVLDS | 76 | EmMIC2 |
| | M | | | | RWRYVPY | | |
| NBX0708 | KRTADRY | 27 | ISGNGLVR | 52 | AADFVLDS | 77 | EmMIC2 |
| | VM | | | | RWRYVPY | | |
| NBX0709 | GSIFSRDT | 28 | ITNFGGT | 53 | AAGLVSLH | 78 | EmMIC2 |
| | M | | | | THTWEY | | |
| NBX0710 | GSIFSINA | 29 | ITSGGST | 54 | AADYVGLG | 79 | EmMIC2 |
| | M | | | | DDAFYVPY | | |
| NBX0711 | RSIRSADA | 30 | ITSGGST | 55 | NAVGDRTS | 80 | EmMIC2 |
| | M | | | | Y | | |
| NBX0712 | GRTFTRN | 31 | ISWSGNR | 56 | AAAREAIT | 81 | EmMIC2 |
| | AM | | | | STYYTPHV | | |
| | | | | | LTDYDA | | |
| NBX0713 | GRTFRRN | 32 | ISWSGNR | 57 | AAARERIT | 82 | EmMIC2 |
| | AM | | | | STYYTPHV | | |
| | | | | | LTDYDA | | |
| NBX0714 | GSIFSRDT | 33 | ITNFGGT | 58 | AAELVSLR | 83 | EmMIC2 |
| | M | | | | THTWEY | | |
| NBX0715 | GRTFSRN | 34 | ISWSGNR | 59 | AAARERIT | 84 | EmMIC2 |
| | AM | | | | STYYTPHV | | |
| | | | | | LTDYDA | | |
| NBX0716 | GRTFSTFP | 35 | INWFTTDT | 60 | AAARSNTG | 85 | EmMIC2 |
| | V | | | | TSRFDY | | |
| NBX0717 | TNIATFTT | 36 | TTPWGAT | 61 | NAQQDIPT | 86 | EmMIC2 |
| | M | | T | | TQTY | | |
| NBX0718 | GRTFSSYA | 37 | VGWSGGR | 62 | AATRGWA | 87 | EmMIC2 |
| | M | | T | | QATLLYDY | | |
| | | | | | DY | | |
| NBX0726 | GLTFGNY | 38 | MSGSGAG | 63 | AANSIYPG | 88 | EtMIC2 |
| | AM | | T | | RRWASYDY | | |
| NBX0727 | GLTLGNY | 39 | MSGSGAG | 64 | AANSIYPG | 89 | EtMIC2 |
| | AL | | T | | RRWASYDY | | |
| NBX0728 | ASIFSRDT | 40 | VTNFGGT | 65 | AADLISLRT | 90 | EtMIC2 |
| | M | | | | HTWEY | | |
| NBX0731 | GFTFSRDT | 41 | ITNFGGT | 66 | AAGLVSLR | 91 | EmMIC2 |
| | M | | | | THTWEY | | |
| NBX0732 | GRTFRRN | 42 | ITWSGNR | 67 | AAARERIT | 92 | EmMIC2 |
| | AM | | | | STYYTPHV | | |
| | | | | | LTEYDA | | |
| NBX0733 | GSIFSVDA | 43 | FTKGGST | 68 | NAVGDRTGA | 93 | EmMIC2 |
| | M | | | | | | |
| NBX0734 | GSIFSRDT | 44 | ITNFGGT | 69 | AAGLVSLR | 94 | EmMIC2 |
| | M | | | | THTWEY | | |
| NBX0740 | GRTFVNY | 45 | INWSGGS | 70 | AAEVGYG | 95 | EmMIC2 |
| | NM | | T | | YQGPPLTT | | |
| | | | | | PSMYDY | | |
| NBX0748 | GSIFSRDT | 46 | ITNFGGT | 71 | AAGLLSLR | 96 | EtMIC2 |
| | M | | | | SHTWEL | | |
| NBX0749 | GSIFSRDT | 47 | ITNFGGT | 72 | AAGLLSLR | 97 | EtMIC2 |
| | M | | | | THTWEY | | |
| NBX0750 | GRTFSGY | 48 | ITWSGDST | 73 | AVKSQTYS | 98 | EmMIC2 |
| | VT | | | | TDYVQPRR | | |
| | | | | | YAY | | |
| NBX0751 | GTIFSITP | 49 | ISGGGST | 74 | NAASLAIV | 99 | EmMIC2 |
| | M | | | | RGINNY | | |
| NBX0752 | GSIFSRDN | 50 | ITNFGGT | 75 | AAGLVSLL | 100 | EmMIC2 |
| | M | | | | THTWEY | | |
| NBX16011 | GNTFSSYT | 168 | LTKGGST | 225 | NMKTAWTI | 282 | EmAMA1 |
| | M | | | | GTRGYDY | | |
| NBX16012 | RSTFSSYT | 169 | ITNGGST | 226 | NMKTAWTI | 283 | EmAMA1 |
| | M | | | | GTRGYDY | | |
| NBX16013 | GSSISSYA | 170 | ISSGGAT | 227 | NMRQLPR | 284 | EmAMA1 |
| | M | | | | WSQLLGS | | |
| NBX16014 | GSTISSYA | 171 | ISSGGGT | 228 | NMRQLPR | 285 | EmAMA1 |
| | M | | | | WSLLLGA | | |
| NBX16015 | GSTLTRYS | 172 | ILKGGST | 229 | HLDWTDF | 286 | EmAMA1 |
| | V | | | | | | |
| NBX16016 | GSSFSSYA | 173 | IGSLGSP | 230 | NMRLLTT | 287 | EmAMA1 |
| | M | | | | WYNLLGS | | |
| NBX16017 | RNIFGVA | 174 | VSSSGTT | 231 | RIATNVPP | 288 | EmAMA1 |
| | HM | | | | YNY | | |
| NBX16018 | GSSISNYA | 175 | ISQRMDT | 232 | NIRILPTWA | 289 | EmAMA1 |
| | M | | | | TTVGS | | |
| NBX16019 | GGSITTNR | 176 | IAYDGST | 233 | TRGGDYSS | 290 | EmMIC1 |
| | YYW | | | | NDYYGMDY | | |
| NBX16020 | GSSLSRYS | 177 | LTSRGDN | 234 | HLDWTDF | 291 | EmAMA1 |
| | V | | | | | | |
| NBX16021 | GSDVSTY | 178 | ISRGGDT | 235 | NIRILPNW | 292 | EmAMA1 |
| | AL | | | | ATTVGR | | |
| NBX16022 | GSSGSDY | 179 | ISRGGDT | 236 | NMRILPNW | 293 | EmAMA1 |
| | AL | | | | NPIVGR | | |
| NBX16023 | VRTFSNY | 180 | ISSNGRT | 237 | NMRLTTS | 294 | EmAMA1 |
| | AM | | | | WERLLGA | | |
| NBX16024 | GSTFSTFS | 181 | ISSGGST | 238 | NMRQLPS | 295 | EmAMA1 |
| | RYAM | | | | WSQLLGS | | |
| NBX16025 | GDSFILYT | 182 | ITSGGTT | 239 | NAKKVRS | 296 | EmAMA1 |
| | A | | | | WPITEEPR | | |
| | | | | | DY | | |
| NBX16026 | GSTLTRYS | 183 | ILKGGST | 240 | HLDWTDF | 297 | EmAMA1 |
| | V | | | | | | |
| NBX16027 | GSSISSYA | 184 | IGSGGTT | 241 | NMRLSTLS | 298 | EmAMA1 |
| | M | | | | LHRALGA | | |
| NBX16028 | GSTFTSSG | 185 | ISSGGST | 242 | NIRILPRWD | 299 | EmAMA1 |
| | STFSRYA | | | | QTVGS | | |
| | M | | | | | | |
| NBX16029 | GSIFTAGS | 186 | ISSSGST | 243 | NMRQLPQ | 300 | EmAMA1 |
| | TFNTYAM | | | | WSTLLGA | | |
| NBX16030 | GNTYNAH | 187 | MDFNGES | 244 | KGSWYFLG | 301 | EmMIC1 |
| | TM | | | | DY | | |
| NBX16033 | GSTFSSYS | 188 | IRSNDST | 245 | NFWDGSFN | 302 | EtRON2 |
| | M | | | | QY | | |
| NBX16034 | GSDTSVY | 189 | ITSGGTV | 246 | NAERVLAR | 303 | EtRON2 |
| | AM | | | | QNY | | |
| NBX16035 | GSIFSINVI | 190 | SGSTT | 247 | NAVVTSRT | 304 | EtRON2 |
| | | | | | SLGLRSYV | | |
| | | | | | Y | | |
| NBX16036 | GNIGWIN | 191 | ISSAEAT | 248 | HTRRWYD | 305 | EtRON2 |
| | DM | | | | DGYDYDT | | |
| NBX16037 | GSIFSINVI | 192 | SDRTT | 249 | NAVVTSRT | 306 | EtRON2 |
| | | | | | SQGLRSYAY | | |
| NBX16038 | RSTISGYG | 193 | LSSGGMT | 250 | NTFDGA | 307 | EtRON2 |
| | G | | | | | | |
| NBX16039 | GIIFSFSLL | 194 | MSTAGNT | 251 | NLRRSWAP | 308 | EtRON2 |
| | NV | | | | SDSGF | | |
| NBX16040 | GTTITINT | 195 | IVPSGKT | 252 | NSDGRFRG | 309 | EtRON2 |
| | M | | | | IGTY | | |
| NBX16041 | GSIFSINVI | 196 | SGSTT | 253 | NAVVTSRT | 310 | EtRON2 |
| | | | | | SLGLRSYAY | | |
| NBX16042 | GITFNFNY | 197 | ISDGGNT | 254 | NLRRSWAP | 311 | EtRON2 |
| | YVM | | | | ADNGH | | |
| NBX16043 | GIAFSMFD | 198 | VSDGGST | 255 | KAARSWA | 312 | EtRON2 |
| | M | | | | YGSGY | | |
| NBX17001 | GITPSRHP | 199 | LTDADST | 256 | FAILGGRS | 313 | EtAMA1 |
| | M | | | | Y | | |
| NBX17002 | QSITSTYG | 200 | INHTGNT | 257 | SPAVWTGL | 314 | EtAMA1 |
| | M | | | | RP | | |
| NBX17003 | GSTPSRHP | 201 | ITNVDST | 258 | FAVLDGRS | 315 | EtAMA1 |
| | M | | | | Y | | |
| NBX17004 | VSTTSTYG | 202 | INHTGGT | 259 | SPPVWTGL | 316 | EtAMA1 |
| | M | | | | RP | | |
| NBX17005 | GTIFRFTV | 203 | ITYTDAT | 260 | FAHYAGY | 317 | EtAMA1 |
| | M | | | | Y | | |
| NBX17006 | GITSSTYA | 204 | MASSGST | 261 | KVPRFGGS | 318 | EtAMA1 |
| | M | | | | DY | | |
| NBX17007 | GITSSTYA | 205 | IASSGST | 262 | KVPRYGGS | 319 | EtAMA1 |
| | M | | | | DY | | |
| NBX17008 | RSSLSTYA | 206 | ITTGGVT | 263 | VRSPRTSW | 320 | EtMIC1 |
| | M | | | | TS | | |
| NBX17009 | ASALRM | 207 | LDNAGKT | 264 | QAHRWTF | 321 | EtMIC1 |
| | | | | | DGWQDY | | |
| NBX17010 | RSTLSTYA | 208 | ITTGHIT | 265 | VRSPRTSW | 322 | EtMIC1 |
| | M | | | | TS | | |
| NBX17011 | ASALRM | 209 | IDNAGKT | 266 | QAHRWTF | 323 | EtMIC1 |
| | | | | | DGWQDY | | |
| NBX17012 | ASALRM | 210 | MDNAGST | 267 | QARRWTF | 324 | EtMIC1 |
| | | | | | DGWQDY | | |
| NBX17013 | GSTLSTYA | 211 | ITTGRVT | 268 | VRSPRTPW | 325 | EtMIC1 |
| | M | | | | SS | | |
| NBX17014 | SSLSTYA | 212 | ITTGHIT | 269 | ARSPRTSW | 326 | EtMIC1 |
| | M | | | | TV | | |
| NBX17015 | GFSLASY | 213 | IGYNSGW | 270 | AARWSFG | 327 | EtMIC1 |
| | HI | | T | | GQCSYGTH | | |
| | | | | | NVQRY | | |
| NBX17016 | RPVFSM | 214 | TTNGKEP | 271 | KARHWEF | 328 | EtMIC1 |
| | | | | | DGVKEY | | |
| NBX17017 | GRAFSTY | 215 | ITSSGNI | 272 | NRGVLSPS | 329 | EtAMA1 |
| | HM | | | | DVYWPSTT | | |
| NBX17018 | GSIFRFTV | 216 | ITYPGGT | 273 | AHYGSYY | 330 | EtAMA1 |
| | M | | | | | | |
| NBX17019 | GSTFSSYA | 217 | ISSSGEIT | 274 | AIGTMA | 331 | EmRON2 |
| | V | | | | | | |
| NBX17020 | GRPFGSY | 218 | MTSAGSG | 275 | WSALGY | 332 | EmRON2 |
| | VM | | GVA | | | | |
| NBX17021 | RSALSM | 219 | KDNAGVT | 276 | QARRWTL | 333 | EtMIC1 |
| | | | | | DGWQDY | | |
| NBX17022 | ASALRM | 220 | IDSAGNT | 277 | QARRWTF | 334 | EtMIC1 |
| | | | | | DGWQDY | | |
| NBX17023 | RSTLSTYA | 221 | ITTGRIT | 278 | ARSPRTSW | 335 | EtMIC1 |
| | M | | | | IL | | |
| NBX17024 | GGSITTSY | 222 | IADDGSA | 279 | ARRGGAW | 336 | EmRON2 |
| | STW | | | | GSNWWAY | | |
| | | | | | HMDD | | |
| NBX17034 | GSITDDHY | 223 | IAAAGNI | 280 | ARYLKLGL | 337 | EtMIC1 |
| | YF | | | | SGMDY | | |
| NBX17035 | GFTFSAA | 224 | IYSDGTRT | 281 | SRDNFGLG | 338 | EtMIC1 |
| | YM | | | | DY | | |

Antibodies Recombinantly Expressed

In another aspect, the present invention provides a method for producing V_{H}H in a suitable producing organism. Suitable producing organisms include, without limitation, bacteria, yeast and algae. In certain embodiments, the producing bacterium is Escherichia coli. In certain embodiments, the producing bacterium is a member of the Bacillus genus. In certain embodiments, the producing bacterium is a probiotic. In certain embodiments, the yeast is Pichia pastoris. In certain embodiments, the yeast is Saccharomyces cerevisiae. In certain embodiments, the alga is a member of the Chlamydomonas or Phaeodactylum genera.

Antibodies Added to Feed

In yet another aspect, the present invention provides a polypeptide or pluralities thereof comprising a V_{H}H or V_{H}Hs that bind disease-causing agents and are administered to host animals via any suitable route as part of a feed product. In certain embodiments, the animal is selected from the list of host animals described, with that list being representative but not limiting. In certain embodiments, the route of administration to a recipient animal can be, but is not limited to: introduction to the alimentary canal orally or rectally, provided to the exterior surface (for example, as a spray or submersion), provided to the medium in which the animal dwells (including air based media), provided by injection, provided intravenously, provided via the respiratory system, provided via diffusion, provided via absorption by the endothelium or epithelium, or provided via a secondary organism such as a yeast, bacterium, algae, bacteriophages, plants and insects. In certain embodiments, the host is from the superorder Galloanserae. In certain embodiments, the host is a poultry animal. In certain embodiments, the poultry animal is a chicken, turkey, duck, quail, pigeon, squab, pheasant or goose. In certain embodiments, the poultry animal is a chicken.

Feed Product

In a further aspect, the present invention provides a polypeptide or pluralities thereof comprising a V_{H}H or V_{H}Hs that bind disease-causing agents and are administered to host animals in the form of a product. The form of the product is not limited. In certain embodiments, the product is feed, pellet, nutritional supplement, premix, therapeutic, medicine, or feed additive, but is not limited to these forms.

Feeding Dosage

In a further aspect, the present invention provides a polypeptide or pluralities thereof comprising a V_{H}H or V_{H}Hs that bind disease-causing agents and are administered to host animals as part of a product at any suitable dosage regime. In practice, the suitable dosage is the dosage at which the product offers any degree of protection against a disease-causing agent, and depends on the delivery method, delivery schedule, the environment of the recipient animal, the size of the recipient animal, the age of the recipient animal and the health condition of the recipient animal among other factors. In certain embodiments, V_{H}Hs are administered to recipient animals at a concentration in excess of 1 mg/kg of body weight. In certain embodiments, V_{H}Hs are administered to recipient animals at a concentration in excess of 5 mg/kg of body weight. In certain embodiments, V_{H}Hs are administered to recipient animals at a concentration in excess of 10 mg/kg of body weight. In certain embodiments, V_{H}Hs are administered to recipient animals at a concentration in excess of 50 mg/kg of body weight. In certain embodiments, V_{H}Hs are administered to recipient animals at a concentration in excess of 100 mg/kg of body weight. In certain embodiments, V_{H}Hs are administered to recipient animals at a concentration less than 1 mg/kg of body weight. In certain embodiments, V_{H}Hs are administered to recipient animals at a concentration less than 500 mg/kg of body weight. In certain embodiments, V_{H}Hs are administered to recipient animals at a concentration less than 100 mg/kg of body weight. In certain embodiments, V_{H}Hs are administered to recipient animal at a concentration less than 50 mg/kg of body weight. In certain embodiments, V_{H}Hs are administered to recipient animals at a concentration less than 10 mg/kg of body weight.

Feeding Frequency

In a further aspect, the present invention provides a polypeptide or pluralities thereof comprising a V_{H}H or V_{H}Hs that bind disease-causing agents and are administered to host animals as part of a product at any suitable dosage frequency. In practice, the suitable dosage frequency is that at which the product offers any protection against a disease-causing agent, and depends on the delivery method, delivery schedule, the environment of the recipient animal, the size of the recipient animal, the age of the recipient animal and the health condition of the recipient animal, among other factors. In certain embodiments, the dosage frequency can be but is not limited to: constantly, at consistent specified frequencies under an hour, hourly, at specified frequencies throughout a 24-hour cycle, daily, at specified frequencies throughout a week, weekly, at specified frequencies throughout a month, monthly, at specified frequencies throughout a year, annually, and at any other specified frequency greater than 1 year.

Feed Additives

In a further aspect, the present invention provides a polypeptide or pluralities thereof comprising a V_{H}H or V_{H}Hs that bind disease-causing agents and are administered to host animals as part of a product that also comprises other additives or coatings. In practice, the most suitable coating or additive depends on the method of delivery, the recipient animal, the environment of the recipient, the dietary requirements of the recipient animal, the frequency of delivery, the age of the recipient animal, the size of the recipient animal, the health condition of the recipient animal In certain embodiments, these additives and coatings can include but are not limited to the following list and mixtures thereof: a vitamin, an antibiotic, a hormone, an antimicrobial peptide, a steroid, a probiotic, a probiotic, a bacteriophage, chitin, chitosan, B-1,3-glucan, vegetable extracts, peptone, shrimp meal, krill, algae, B-cyclodextrin, alginate, gum, tragacanth, pectin, gelatin, an additive spray, a toxin binder, a short chain fatty acid, a medium chain fatty acid, yeast, a yeast extract, sugar, a digestive enzyme, a digestive compound, an essential mineral, an essential salt, or fibre.

Non-Feed Uses

In a further aspect, the present invention provides a polypeptide or pluralities thereof comprising a V_{H}H or V_{H}Hs that bind disease-causing agents, and can be used in a non-feed use, such as but not limited to: a diagnostic kit, an ELISA-based assay, a western blot assay, an immunofluorescence assay, or a FRET assay, in its current form and/or as a polypeptide conjugated to another molecule. In certain embodiments, the conjugated molecule is can be but is not limited to: a fluorophore, a chemiluminescent substrate, an antimicrobial peptide, a nucleic acid or a lipid.

Antigens

In a further aspect, the present invention provides a polypeptide or pluralities thereof comprising a V_{H}H or V_{H}Hs that bind disease-causing agents, produced by a species of Eimeria. In certain embodiments, the species does not belong to the Eimeria genus but is capable of harbouring disease-causing agents shared by Eimeria species. In certain embodiments, the Eimeria species refers to both current and reclassified organisms. In certain embodiments, the Eimeria species is Eimeria tenella. In certain embodiments, the Eimeria species is Eimeria maxima.

In certain embodiments, the V_{H}H or plurality thereof is capable of binding to one or more disease-causing agents, originating from the same or different species. In certain embodiments, the disease-causing agent is a polypeptide with 80% or greater amino acid sequence identity to Eimeria maxima MIC1 (EmMIC1, SEQ ID 101). In certain embodiments, the disease-causing agent is a polypeptide with 80% or greater amino acid sequence identity to Eimeria tenella MIC1 (EtMIC1, SEQ ID 102). In certain embodiments, the disease-causing agent is a polypeptide with 80% or greater amino acid sequence identity to Eimeria maxima MIC2 (EmMIC2, SEQ ID 103). In certain embodiments, the disease-causing agent is a polypeptide with 80% or greater amino acid sequence identity to Eimeria tenella MIC2 (EtMIC2, SEQ ID 104). In certain embodiments, the disease-causing agent is a polypeptide with 80% or greater amino acid sequence identity to Eimeria maxima AMA1 (EmAMA1, SEQ ID 105). In certain embodiments, the disease-causing agent is a polypeptide with 80% or greater amino acid sequence identity to Eimeria tenella AMA1 (EtAMA1, SEQ ID 106). In certain embodiments, the disease-causing agent is a polypeptide with 80% or greater amino acid sequence identity to Eimeria maxima RON2 (EmRON2, SEQ ID 107). In certain embodiments, the disease-causing agent is a peptide with 80% or greater amino acid sequence identity to a peptide from EmRON2 that binds EmAMA1 (SEQ ID 108). In certain embodiments, the disease-causing agent is a polypeptide with 80% or greater amino acid sequence identity to Eimeria tenella RON2 (EtRON2, SEQ ID 109). In certain embodiments, the disease-causing agent is a peptide with 80% or greater amino acid sequence identity to a peptide from EtRON2 that binds EtAMA1 (SEQ ID 110). In certain embodiments, the disease-causing agent is an exposed peptide, protein, protein complex, nucleic acid, lipid, or combination thereof, that is associated to the surface of the Eimeria parasite. In certain embodiments, the disease-causing agent is an exposed peptide, protein, protein complex, nucleic acid, lipid, or combination thereof, that is deposited on the surface of host cells by the Eimeria parasite. In certain embodiments, the disease-causing agent is the Eimeria parasite.

Antigen Sequences
EmMIC1
>AAA29076.1 em100 gene is homologous the Eimeria tenella gene et100
(accession number M73495) encoding the microneme protein Etp100
[Eimeria maxima]
(SEQ ID 101)
MALLPTQRLAPGWALSLLVFLAAGLTFHSSHAAASSEADQVCTRLLDVMLVVD
ESGSIGTSNYGKVRSFISNFAGTMPLSPDDVRVGLVTFGTSAVTRWDLSDSRAQNADLL
AAAAKKLPYAAGSTYTHLGLAKAEEILFSFQKGGRDNAPKMILVMTDGASSRRSQTLS
AAEKLRNRGVIIVVLGVGTGVNSAECRSIAGCDTSDTVECPRYLQSNWGGVSSQINGIIK
AACKDLAKDAVCSEWSEYGPCEGECGTEGTRTSTRVEIAPPRPGTPPCPTCEAPQGRSC
AQQPPGLMRTEQCTMPACKIDAHCGDFGPWSEWSTTCGSATRQRVRQGYEDPPASGG
GLSCIDQNPPKYAKEVEVVQKSPCPVQQQPGPWSDWSDCSATCGGGTRYREREGYPQE
GELFGGQTLQAQGLDVRETDTCNENPCPVDATCGEWTEFSDCSRVCGGGTKERRREP
WLDNAQFGGRSCSQQHPEGPTESVECNEHPCPVDEVVGEWEDWGPCSEQCGRGRQFR
YRGPSLQQAMFGGKTIEEQNAGVPEEQKILKVEERPCNDVPCGPCTLPFTEWTACESCS
GTRTRDSMVAFDYDDRQCQNPTHEEESCDAVCEESASGGGVGGGAGGAGGGGGGSA
GGEGSNGAGPGEDKEEESKGFPTAAVAGGVAGGVLAIAAGAGAFYGLSGGAASAAGG
AAAEVMVESGTANPPEVEKESLITAGEQSEMWAS
EtMIC1
>AIR96017.1 microneme protein Mic-1 [Eimeria tenella]
(SEQ ID 102)
MAPLPRRRLAPCRALSLLVGLLAASFAFSSLQPGATTSSGQDQVCTSLLDVMLV
VDESGSIGTSNFRKVRQFIEDFVNSMPIPPEDVRVGLITFATRSKVRWNLSDPKATNPSL
AISAARSLSYSTGVTYTHYGLQDAKKLLYDTNAGARNNVPKLVLVMTDGASNLPSQTR
SSAAALRDAGAIVVVLGVGSGVNSSECRSIAGCSTSNCPRYLQSNWSNVTQQVNGIIKA
ACKDLAKDAVCSEWSEYGPCVGECGKEGVQTSTRVEISPQKPGSPPCPTCEAPRGRSCA
EQPPGLTRTQPCTMPVCKTDAHCGEFGAWSEWSTTCGTATRKRQREGYNSPPAAGGG
LSCMEQNPPKHEFEVETVQKSPCPVQQQPGPWSEWTECSATCGGGTKHREREGLPQEG
ELYGGQTLEQQGIAVRETASCSENPCPIGATCGEWTEYSACSRTCGGGTQERKREPWLD
NAQHGGRTCMEQYPDGPISVRECNTQPCPVDEVVGDWEDWGQCSEQCGGGKRTRNR
GPSKQEAMFGGKTVAQQNVELPEGEKIEVVQEGGCNEVPCGPCTLPFSEWTECESCSG
HRTRESAVAFDYTDRMCSGDTHEVHSCEEYCSQNAGGGAGGDGGAGGGTGGSGEEEG
KEESSGFPTAAVAGGVAGGVLAIAAGAGAFYGLSGGSAAAATEAGAEVMTEAGTSNA
AEVEKESLISAGEQSEMWAS
EmMIC2
>CBX60033.1 Microneme protein 2 [Eimeria maxima]
(SEQ ID 103)
MARALSLIALGLLFSLPSTSAVRTKVPGDEPSGPDSALTAGSPTRGNHGVGGFAE
AHCNRLTVRGGLQEKEAVKVTANGWGKGDADFFVELVTDNTRGMLQIRESQTEAGPG
LAGGGGQEGGSTEGSKTDEGPVKEQPSIPIVGVRIPGSANENGESRKPAVLVYGEGESAP
KEFPLDSPAGPTSPFMVVLQQKSPTEMTVRLFTWIPNGSGGDGSWHETFVDVGVGINHR
DVMVVVSDCVPHSLRIYGSSSADLVTADEKTCQATEPQLVNLTSPPENRTHPGAETSTT
TSVSS
EtMIC2
>AAD05559.1 microneme protein Etmic-2 [Eimeria tenella]
(SEQ ID 104)
MARALSLVALGLLFSLPPSSAVRTRVPGEDSFSPESGVLSGTDAPERRPIVPGLVE
GNCGRLTVRNGPSVDETIKVTSAGWTKSERDFIVSLVADETRKVVQLRESEGASGASGP
GPAPAEKPPSGQGSAEEAPKGEGGQEKPSVPLIAVRIHGSGGDKGESAPQSAVLLYGND
ESEPTEVPLETAAGPTTPLMVLITQQNPKEVEVRVLAWISTDATTGKGSWKENSVVVGS
SLSGRDLTVNLSDCGPSSLRVYGSASADLVTVKEGMCEADDPELIALTRPHTSAASPLP
AEEGDVAQDAQQSAGAQQEAEAQEVGEPQQEAAAAEQGSSAAESDTQQSS
EmAMA1
>SNT95431.1 Apical membrane antigen 1 [Eimeria maxima]
(SEQ ID 105)
MCGLRAAFTEAVCLGLLSLGSTVVQGIKDKVHQGHTEAAATAAAGNLSAELHA
ALHQPNDNPFLVPPLSDFMDRFNIPKVHGSGIYVDLGGDKEVDGRTYREPSGLCPVFGK
TIVLYQPQNNPNYKNDFLDDMPTKQQSDAVGHPLPGGFNNSFKMPDKSPYSPMSAQKL
NSYKQLKANTPMGKCAEMSFMTTAGKNSSYRYPWVYDTKRDLCYFLYLPVQRLMGE
RYCSVDGKPDGMTWYCFEPHKALDSRPELVYGSAYVGRDPDYWETHCPNKAVKDAV
FGVWVSGRCTEHKHLDGAKKEKVNSKAECWSLAFENPEVASDHPVTEDENFGTYGYF
FPSTEPNQPKSGGEGVNFASFYPGSMECWLSGEIPTCLVPLEGAAAFTALGSLEEETAPC
TDSFPQTKTPCDRNTCTQIVATCVSGTLVSEEVPCSPEDGTRCEGGFPKGVMIGLAAAG
GILLLLLGGGGFLLYRSRMRPAAKGDEATRSDYVQEEAAANRRKQRQSDLVQQAEPSF
WEEAEADEAETGESTHVLVDQDY
EtAMA1
>XP_013229486.1 apical membrane antigen-1 [Eimeria tenella]
(SEQ ID 106)
MRRLSPALGLLAAALSCAGPAAGVQHKLQHRQQQQQQHSHASTSHAAAVLAA
SSDASTDSNPFMQPPYAEFMARFNIPKVHGSGVYVDLGNDKEVKGKMYREPGGRCPVF
GKNIEFYQPLDSDLYKNDFLENVPTEEAAAAAKPLPGGFNNNFLMKDKKPFSPMSVAQ
LNSYPQLKARTGLGKCAEMSYLTTAAGSSYRYPFVFDSKKDLCYLLLVPLQRLMGERY
CSTRGSPPGLSHFCFKPLKSVSLRPHLVYGSAYVGERPDDWETKCPNKAVKDAVFGVW
EGGRCEEQRLRLGAQTAAAAAKEDCWALAFNNPFAASDQPTSQDEAATSPGYYFPSIT
PSQPKSGGVGVNFASYYPSGECVLSGEVPTCLLPRQGAAAFTSVGSLEEEELPHCDPTFP
ASLGSCDPSSCKAILTECRGGRLVEQQTDCVPEDGSKCESKGGGVFIGLAVAGGLLLLL
LTGGAFFIYKQRQKALPKESSPQRTDFVQDEAATGRGKKRQSDLVQQAEPSFWEEAEA
DEPHADENTQVLLDQEY
EmRON2
>XP_013337434.1 hypothetical protein, conserved [Eimeria maxima]
(SEQ ID 107)
MFRLLYLPGLVTILSVSQRTPEVKITMLGAFSIFALSTLLSLPPYSWRMAAMADS
LSVTPEYEVEGPTNFLTPSLVTLDSALEGLDSGGTPGFSGQYADLLDRICPADSPALDLP
QQPTREQNIGELELMLSDNDLGEATNKLWLAFYGHEVPKAASSVEELSAQFLELVGQV
RVAFQDVHHHMVKQEGHPEFHNSQLPRSIVPIVGARNPLMLGLFWNLLIAYTGFDSYF
GEDSITLPFFSWPSLLASLGGQSSSHIDAMCSVQRSRSLTEKFFKWRSPRGIQQNRRHKR
VSSLRESSHRTFCELIDRLISSLGEFVAGHVTTLAAAGVPVELGISPLQNMKRLHAETCLP
GEDGHILAPCIFEESRLALDSQQLVKMEDDTDQQIRHSAQAELLKQAHTTISGLGALDC
KLSSRKWRALPYTPMPRLFKLPELAQLRKDWILERLNLAVATMLTTSSLDNASNYWLA
FDRRNFKRAAQDFKKHTIDVLRSVGMEAIGRRLLQSSGSEISDDEVQAAASRLKDLRAV
SDGMRVALALYAILNAGRHAQSAHVGSQGHAIHAQCYGTCCSTAEPYAYVGVRVPEL
SGRDAQEHERNQDPESQKGAWSKIDLNGLPLPDLTDWWSRLNYIVVQALESYLVLEKH
LEFLEDTVFTLVSDSLRKLKADAGQTLFFTRIAHQKRQGPLQRLWEGAKKSLMGFLYRS
PGRQHGIWFGVTVDFEQLHDLLGQLKLVIEAEPRLTIKINMQEALLREMETRIRVEGSDI
SRVPPLVERNMGMLGVRRAYGTLSEGLRDTEFQRSMCIDHCRGLWQMALSTMLPSML
SPDIFRKYEKAFGTPWALEHLSDPALVNSRRMVLKSDAALNFFDHSTPKEVREELKGLE
SGQASMFAYYMLFSSRAHQVLGNHYLGLYLRQQAPFMGNMVVDWISTRRKHAVAAI
VSSFVLTFMGIYAVMSFVDILQNLIVSGAPPPFDCLWNPVFGEMACSPVPGGASLGTAW
ATAIEQVFLIGLFSGTAGGFSLFLTINSAIAVVVNQSKTLMRLQMCLGSAISRLLRRGKRS
FSRIREYFIKRSSVKRVMLQRAVAGMKAGSSTATMSNSETLEAADQLLDRLTRTGRANP
LKASGGKPAFK
EmAMA1-binding EmRON2 peptide
SEQ ID 108)
DILQNLIVSGAPPPFDCLWNPVFGEMACSPVPGGASL
EtRON2
>XP_013231132.1 rhoptry neck protein 2, putative, partial
[Eimeria tenella]
(SEQ ID 109)
MGVFSRLLSAALLCAAASPALLSPQAAAAAAAPAEGSTSFTLPSGGSLHVQVLR
GPQQQQQQQQQGPWGSTMSFAGPTGATGGYSVTDSATLRSSAGPPAAAAAAAAAEGV
YELPTPPTPENPISKTQVLFGWVPEAAAAASLQAVNAEELNNALRSQAFVRKLRTRALV
EKSCMIPLASDRRSYLEAFWSAAAAERKFRAEAESQRAAIEEELKQQQMAAPDMSYAR
SVATRANIESGASPAAAAAAAAGAAAAAAAGEEAAAAALRYQETVDRVLANRIEVLM
TCKLAALLGQPGIFLNDKIAAAEVLDLVATALGIGNTKEEAAAAAASPIVPMFGGLEPFL
LASNPLIVGHVLTLLIAHIDREAFFGEAARKAFYSFTSLAAAAGGEGAVGMLDEMCDRD
RGPRLAAPLLGKYRPKGRGGKPRKRGSSSPWFVRGASRVHRNKLQPELLRAYCDATE
MILNALMLKQEDVQQEMLKFSLPVEPLVDPATNASRIQTKTCRGGAPVCEFENSILSPIA
NPLDPQVLEQNTNTRAAFNLYAGLASAQLGNLLEETGSKYYDVRADQWLEIISKPTAH
SDILEKVFFYDNREFLASKRSKLIKSFETNAKKIAALAGKPAVSISEAAAAYEEIQRSVGS
QTGKKGAAAAKKNLFPTAATLLLKSTLACDMTSSFAAKLESFTQFMFKAKAASGRRQA
PLQRTLMAFIRTGKGSALQSMCSYDPLTFNYLFLYRFVLLGSDPAKTHDKQHAAVSKTP
LTTRLLGSTWTPSILKRVLRGVKRKSSILKAKQLLLESLDAGVFPLLLTSFEWIVHTQAA
LQVNQNSEMFHELEAQQPRALPAEPQKAAEAKLHEGGLVKRTDEQLAEWAEFGVPAA
LREQLRRGEKLPKAVALERIPTPDLTQWEQQLNRKWLAALEAYLRHPYGAAAAAARD
PVALLVQRSRDRLEAELDSTIFLGRIVGAPRGPRRGRRALRAVGGLFRALLRAPRQSEFA
VWMGVKVHADRVLRVLHAVHAAAEVAKTAGLFEHVREAFLELVRDAVLGSLQNQLR
VPGFDTFAAVEAGLRGGEVSAAAAAAAAAAAAARRSAGFLSVHPELAALGPAAREAE
FQNSMCMDHCEALWTLVTSLVFSALQNPRKWRDYEKEVGGAAAAAALADPRRVNSF
RLGLSVQTDFFENVLDKKSKRNIQKMKFGGGSWFAYALLLAARLHRGLGHSDLATFFS
FQAPYLGHFVLQWQQQRREARRKALLSMLSLGFFFAYTFISVSDITQHLNDSGLGPAVE
CLENLVVGPVCPAAVVAPAVRSAAAAAAADVFKVGLFGLLTPYLVWPMAAAAAWQL
LRSEFKVLLQFEMSLKSLFSRFSSWVRRPFARWWQQRRRLKQLLLQSAAAKFRTEKKR
LHGRDPPPRDF
EtAMA1-binding EtRON2 peptide
(SEQ ID 110)
DITQHLNDSGLGPAVECLENLVVGPVCPAAVVAPAVR

### Examples

The following illustrative examples are representative of the embodiments of the applications, systems and methods described herein and are not meant to be limiting in any way.

While preferred embodiments of the present invention are shown and described herein, it will be obvious to those skilled in the art that such embodiments are provided by way of example only. Numerous variations, changes, and substitutions will now occur to those skilled in the art without departing from the invention. It should be understood that various alternatives to the embodiments of the invention described herein may be employed in practicing the invention.

Production of Antigens

Recombinant antigens can be purified from an E. coli expression system. For example, an antigen can be expressed at 18° C. in E. coli BL21 (DE3) cells grown overnight in autoinducing media (Formedium). Cells are then lysed by sonication in buffer A (250 mM NaCl, 50 mM CaCl₂, 20 mM imidazole and 10 mM HEPES, pH 7.4) with 12.5 □g/ml DNase I, and 1× Protease inhibitor cocktail (Bioshop). The lysate is cleared by centrifugation at 22000×g for 30 minutes at 4° C., and is then applied to a 5 ml HisTrap HP column (GE Healthcare) pre-equilibrated with buffer A, washed with ten column volumes of buffer A and eluted with a gradient of 0% to 60% (vol/vol) buffer B (250 mM NaCl, 50 mM CaCl₂, 500 mM imidazole and 10 mM HEPES, pH 7.4). The protein is then dialyzed overnight in the presence of TEV against buffer C (250 mM NaCl, 10 mM HEPES, pH 7.4 and 5 mM □-mercaptoethanol) at 4° C. The dialyzed protein is applied to a HisTrap HP column (GE Biosciences) pre-equilibrated with buffer C. 6×His-tagged TEV and 6×His-tag are bound to the column and the antigen is collected in the flowthrough. The sample is dialyzed overnight against buffer D (5 mM NaCl and 10 mM Tris pH 8.8) and then applied to a 5 ml HiTrap Q HP column (GE Healthcare). The protein is eluted with a gradient of 0% to 50% (vol/vol) buffer E (1.0 M NaCl and 10 mM Tris pH 8.8). Lastly, the elution is loaded onto a Superdex 75 Increase 10/300 GL gel filtration column (GE Healthcare) using buffer F (400 mM NaCl and 20 mM HEPES pH 7.4). The protein sample is then concentrated to 1 mg/mL using Amicon concentrators with appropriate molecular weight cutoff (MWCO; Millipore). The purified protein is stored at −80° C.

Alternatively, the EmAMA1-binding peptide of EmRON2 (SEQ ID 108) and the EtAMA1-binding peptide of EtRON2 (SEQ ID 110) were expressed in E. coli as fusions at the C-terminus of glutathione S-transferase (GST). They were purified as described as above without TEV cleavage.

Production of NBXs and Panning

Llama Immunisation

A single llama is immunized with purified disease-causing agents, such as the antigens listed, which may be accompanied by adjuvants. For SEQ ID 108 and 110, the antigenic peptides were provided to the llama as fusions to GST. The llama immunization is performed using 100 μg of each antigen that are pooled and injected for a total of four injections. At the time of injection, the antigens are thawed and the volume increased to 1 ml with PBS. The 1 ml antigen-PBS mixture is then mixed with 1 ml of Complete Freund's adjuvant (CFA) or Incomplete Freund's adjuvant (IFA) for a total of 2 ml. A total of 2 ml is immunized per injection. Whole llama blood and sera are then collected from the immunized animal on days 0, 28, 49, 70. Sera from days 28, 49 and 70 are then fractionated to separate V_{H}H from conventional antibodies. ELISA can be used to measure reactivity against target antigens in polyclonal and V_{H}H-enriched fractions. Lymphocytes are collected from sera taken at days 28, 49, and 70.

Panning

RNA isolated from purified llama lymphocytes is used to generate cDNA for cloning into phagemids. The resulting phagemids are used to transform E. coli TG-1 cells to generate a library of expressed V_{H}H genes. The phagemid library size can be ˜2.5×10⁷ total transformants and the estimated number of phagemid containing V_{H}H inserts can be estimated to be ˜100%. High affinity antibodies are then selected by panning against the antigens used for llama immunization. Two rounds of panning are performed and antigen-binding clones arising from round 2 are identified using phage ELISA. Antigen-binding clones are sequenced, grouped according to their CDR regions, and prioritized for soluble expression in E. coli and antibody purification.

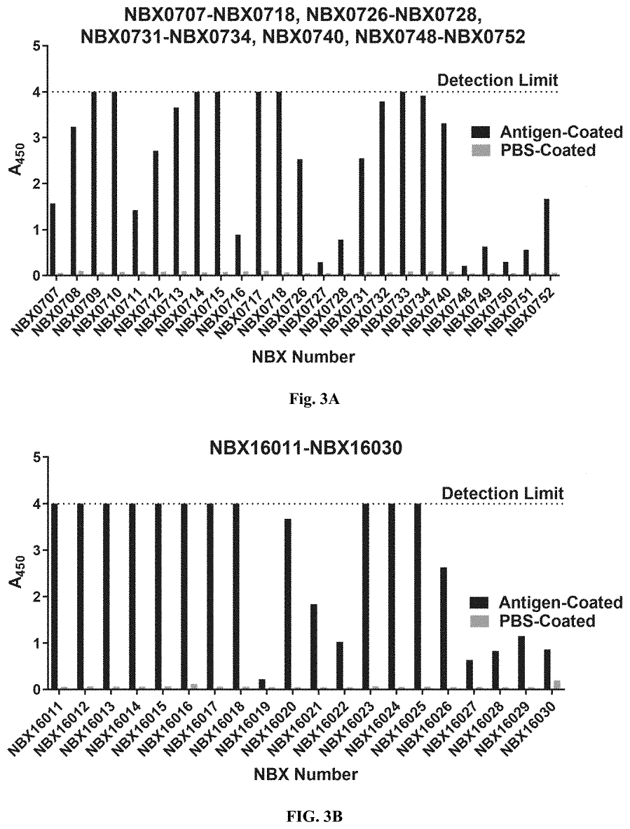

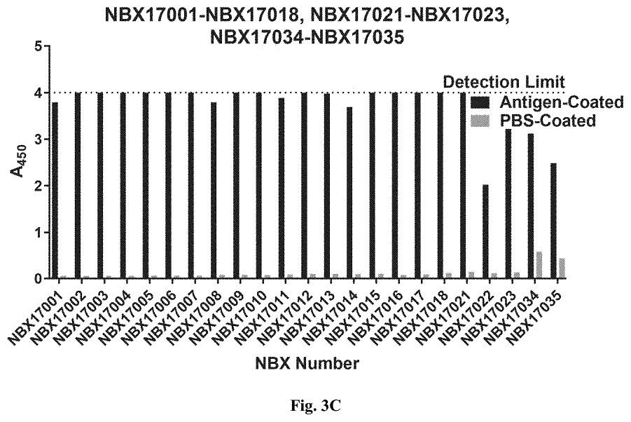

FIG. 3 shows the phage ELISA results for antibodies of this disclosure. Black bars show binding to wells coated with the antigen specified in Tables 1 and 2 dissolved in phosphate-buffered saline (PBS). Grey bars are negative controls that show binding to wells coated with PBS only. In all cases binding to the antigen target is at least four times above binding to the PBS-coated wells. Data for NBX0707-NBX0718, NBX0726-NBX0728, NBX0731-NBX0734, NBX0740, and NBX0748-NBX0752 are shown in panel A. Data for NBX16011-NBX16030 are shown in panel B. Data for NBX017001-NBX017018, NBX17021-NBX17023, and NBX17034-NBX17035 are shown in panel C.

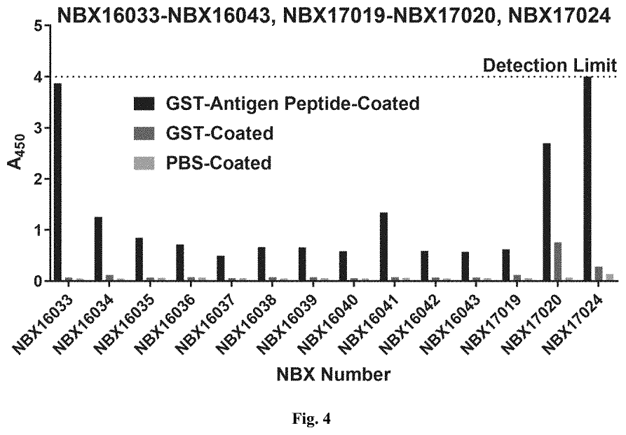

FIG. 4 shows the phage ELISA results for antibodies of this disclosure that target the EmAMA1-binding peptide of EmRON2 (SEQ ID 108) or EtAMA1-binding peptide of EtRON2 (SEQ ID 110). Since the llama was immunized with these peptides as fusions to GST, an additional control was conducted to confirm binding of phage specifically to the RON2 peptide portion of the GST-RON2 peptide fusion proteins. Black bars show binding to wells coated with the GST-RON2 peptide antigen as specified in Tables 1 and 2 dissolved in phosphate-buffered saline (PBS). Dark grey bars are negative controls that show binding to wells coated with GST dissolved in PBS. Light grey bars are negative controls that show binding to well coated with PBS. In all cases binding to the antigen target is at least four times above binding to the PBS-coated wells and at least three times above binding to the GST coated wells.

Purification of V_{H}Hs from E. coli

TEV protease-cleavable, 6×His-thioredoxin-NBX fusion proteins are expressed in the cytoplasm of E. coli grown in autoinducing media (Formedium) for 24 hours at 30° C. Bacteria are collected by centrifugation, resuspended in buffer A (10 mM HEPES, pH 7.5, 250 mM NaCl, 20 mM imidazole) and lysed using sonication. Insoluble material is removed by centrifugation and the remaining soluble fraction is applied to a HisTrap column (GE Biosciences) pre-equilibrated with buffer A. The protein is eluted from the column using an FPLC with a linear gradient between buffer A and buffer B (10 mM HEPES, pH 7.5, 500 mM NaCl, 500 mM Imidazole). The eluted protein is dialyzed overnight in the presence of TEV protease to buffer C (10 mM HEPES, pH 7.5, 250 mM NaCl). The dialyzed protein is applied to a HisTrap column (GE Biosciences) pre-equilibrated with buffer C. 6×His-tagged TEV and 6×His-tagged thioredoxin are bound to the column and purified NBX is collected in the flowthrough. The NBX-containing flowthrough is dialyzed to buffer D (10 mM HEPES, pH 7.0) and applied to a HiTrapSP ion exchange column (GE Biosciences. Highly purified NBX protein is eluted from the column using a linear gradient from buffer D to buffer E (10 mM HEPES, pH 7.0, 500 mM NaCl) NBX proteins are dialyzed overnight to buffer F (20 mM HEPES, pH 7.4, 150 mM NaCl) and concentrated to ˜10 mg/ml.

Purification of V_{H}Hs from P. pastoris

Pichia pastoris strain GS115 with constructs for the expression and secretion of 6×His-tagged V_{H}H are grown for 5 days at 30° C. with daily induction of 0.5% (vol/vol) methanol. Yeast cells are removed by centrifugation and the NBX-containing supernatant is spiked with 10 mM imidazole. The supernatant is applied to a HisTrap column (GE Biosciences) pre-equilibrated with buffer A (10 mM HEPES, pH 7.5, 500 mM NaCl). The protein is eluted from the column using an FPLC with a linear gradient between buffer A and buffer B (10 mM HEPES, pH 7.5, 500 mM NaCl, 500 mM imidazole). NBX proteins are dialyzed overnight to PBS and concentrated to ˜10 mg/ml.

All publications, patent applications, issued patents, and other documents referred to in this specification are herein incorporated by reference as if each individual publication, patent application, issued patent, or other document is specifically and individually indicated to be incorporated by reference in its entirety. Definitions that are contained in text incorporated by reference are excluded to the extent that they contradict definitions in this disclosure.

The following references are incorporated by reference in their entirety.
(1) Van Meirhaeghe, H. & De Gussem, M. (2014). Coccidiosis a major threat to the chicken gut. Retrieved on May 25, 2018 from: https://www.poultryworld.net/Home/General/2014/9/Coccidiosis-a-major-threat-to-the-chicken-gut-1568808W/?dossier=35765&widgetid=1.
(2) Wade, B. & Keyburn, A. (2015). The true cost of necrotic enteritis. Retrieved on May 25, 2018 from: https://www.poultryworld.net/Meat/Articles/2015/10/The-true-cost-of-necrotic-enteritis-2699819W/.
(3) Chapman, H. D. (2014). Milestones in avian coccidiosis research: a review. Poultry Science, 93(3), pp. 501-511.
(4) Boucher, L. E. & Bosch, J. (2015). The apicomplexan glideosome and adhesins-structures and function. Journal of Structural Biology, 190(2), pp. 93-114.
(5) Moore, R. J. (2016). Necrotic enteritis predisposing factors in broiler chickens. Avian Pathology, 45(3), pp. 275-281.
(6) Abid, S. A. et al. (2016). Emerging threat of necrotic enteritis in poultry and its control without use of antibiotics: a review. The Journal of Animal and Plant Sciences, 26(6), pp. 1556-1567.

1. A polypeptide comprising at least one variable region fragment of a heavy chain antibody (V_{H}H), wherein the at least one V_{H}H specifically binds a disease-causing agent, wherein the disease-causing agent is a parasite from the Apicomplexa phylum.

2. The polypeptide of claim 1, wherein the polypeptide comprises a plurality of V_{H}Hs.

3.-5. (canceled)

6. The polypeptide of claim 1, wherein the variable region fragment of the heavy chain antibody comprises an amino acid sequence at least 80%, 90%, 95%, 97%, 98%, 99%, or 100% identical to any one of SEQ ID Nos: 1 to 25 or 111 to 167.

7. The polypeptide of claim 1, wherein the variable region fragment of the heavy chain antibody comprises a complementarity determining region 1 (CDR1) as set forth in any one of SEQ ID Nos: 26 to 50 or 168 to 224, a complementarity determining region 2 (CDR2) as set forth in any one of SEQ ID Nos: 51 to 75 or 225 to 281, and a complementarity determining region 3 (CDR3) as set forth in any one of SEQ ID Nos: 76 to 100 or 282 to 338.

8.-10. (canceled)

11. The polypeptide of claim 1, wherein the Apicomplexan parasite is from the Aconoidasida class, the Gregarinasina subclass, or the Coccidia subclass.

12.-13. (canceled)

14. The polypeptide of claim 1, wherein the disease-causing agent comprises a species of Eimeria.

15. The polypeptide of claim 14, wherein the species of Eimeria is Eimeria tenella or Eimeria maxima.

16. The polypeptide of claim 14, wherein the V_{H}H specifically binds an Eimeria virulence factor.

17. The polypeptide of claim 15, wherein the V_{H}H specifically binds an antigen or polypeptide at least 60%, 70%, 80%, 90%, 95%, 98%, 99%, or 100% identical to SEQ IDs Nos: 101 to 110 or combinations thereof.

18. The polypeptide of claim 16, wherein the Eimeria virulence factor is EmMIC1 polypeptide, EmMIC1-like polypeptide, EtMIC1 polypeptide, EtMIC1-like polypeptide, EmMIC2 polypeptide, EmMIC2-like polypeptide, EtMIC2 polypeptide, EtMIC2-like polypeptide, EmAMA1 polypeptide, EmAMA1-like polypeptide, EtAMA1 polypeptide, EtAMA1-like polypeptide, EmRON2 polypeptide, EmAMA1-binding EmRON2 peptide, EmAMA1-binding EmRON2-like peptide, EmRON2-like polypeptide, EtRON2 polypeptide, EtRON2-like polypeptide, EtAMA1-binding EtRON2 peptide, or EmAMA1-binding EmRON2-like peptide.

19. A nucleic acid encoding the polypeptide of claim 1.

20. (canceled)

21. An expression vector comprising the nucleic acid of claim 19.

22. A cell comprising the nucleic acid of claim 19. of nucleic acids of claim 20.

23.-30. (canceled)

31. The polypeptide of claim 1 further comprising a vitamin, an antibiotic, a hormone, an antimicrobial peptide, a steroid, a probiotic, a probiotic, a bacteriophage, chitin, chitosan, B-1,3-glucan, vegetable extracts, peptone, shrimp meal, krill, algae, B-cyclodextran, alginate, gum, tragacanth, pectin, gelatin, an additive spray, a toxin binder, a short chain fatty acid, a medium chain fatty acid, yeast, a yeast extract, sugar, a digestive enzyme, a digestive compound, an essential mineral, an essential salt, or fibre.

32. A method of producing the polypeptide of claim 1, comprising (a) incubating a cell in a medium suitable for secretion of the polypeptide from the cell; and (b) purifying the polypeptide from the medium.

33. A method of reducing or preventing a poultry-associated parasite infection in a non-human animal comprising administering the polypeptide of claim 1 to the non-human animal.

34. A method for reducing transmission or preventing transmission of a poultry-associated parasite from a poultry species to a non-human animal comprising administering the polypeptide of claim 1 to the poultry species.

35. The method of claim 34, wherein the non-human animal is a species of a chicken, turkey, duck, quail, pigeon, squab, ostrich, pheasant, or goose.

36. The method of claim 34, wherein the non-human animal is a cow, sheep, goat, fish, rabbit, camel, llama, guanaco, alpaca, or vicuna.

37. The method of claim 34, wherein the polypeptide is formulated for introduction to the alimentary canal orally or rectally, provided to the exterior surface, provided to the medium in which the animal dwells, provided by injection, provided intravenously, provided via the respiratory system, provided via diffusion, provided via absorption by the endothelium or epithelium, or provided via a secondary organism such as a yeast, bacterium, algae, bacteriophages, plants and insects to a host.
